SEQUENCE LISTING (15 sequences)

SEQ ID NO: 1 - DNA, 4679 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - protein, 155 aa, adeno-associated virus 2
MVLYLPTSREATDKQLPQMSTHKAFFQAWSGRTEMCTFRGPSGQRFHTRTDIFTPLPSWVDSDLNTLLHRFSSRTPRYLRILRPPSVRQSLLPSSHSTPRDRSAWRSSGSCRRKTANAGIPKFSTLPTTTSLLMWTLLWTLMACIQSLAPLAPDT

SEQ ID NO: 3 - DNA, 181 bp, adeno-associated virus 2
tacggttctgtatctaccaacctccagagaggcaacagacaagcagctaccgcagatgtaaacacacaaggcgttcttccaggcatggtctggcaggacagagatgtgtaccttcaggggcccatctgggcaaagattccacacacggacggacattttcacccctctcccctgatgggtg

SEQ ID NO: 4 - protein, 61 aa, adeno-associated virus 2
YGSVSTNLQRGNRQAATADVNTQGVLPGMVWQDRDVYLQGPIWAKIPHTDGHFHPSPLMGG

SEQ ID NO: 5 - protein, 158 aa, Human papillomavirus type 68
MARFHNPEERPYKLPDLCRTLDTTLHDVTIDCVYCRRQLQRTEVYEFAFGDLHVVYRDGVPLAACQSCIKFYAKIRELRYYSESVYATTLETITNTKLYDLSIRCMCCLKPLSPAEKLRHLNAKRRFHKIAGNFTGQCRHCWTSKREDRRRTRQETQV

SEQ ID NO: 6 - protein, 181 aa, Yarrowia lipolytica
MPNYYLADDTKYTRPSTSAMSNLTELCGLKGLADSMARFDPVTGEKNKLRKSYKNQLLDLTGKFDIPSQPSLVRIIRDPVLAETGGKSLSVFDRDLLSRGLDFDATPSTGIPGFNPAMLGMPGPGKPGIGPQVGGNRLLSVRRSYNTESARESSNNEETPMARRKRKGDDDTDGERRRKRK

SEQ ID NO: 7 - DNA, 4683 bp, Adeno-associated virus 6
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagcgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggttaattcgtgtcagttgaactttggtctcatgtcgttattatcttatctggtcaccatagcaaccggttacacattaactgcttagttgcgcttcgcgaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 8 - protein, 84 aa, Adeno-associated virus 6
MCMLWEPYLEWCGKTETYTCRVLFGPKFLTRMDTFTRLLSWAALDLSTRLLRSSSKTRLFLRILRQSFRLQSLLHSSPSIPQDK

SEQ ID NO: 9 - protein, 53 aa, Adeno-associated virus 6
LNGSCRKKTANAGIPKCSIHLTMQNLPTLISLWTTMDFILSLAPLAPVTSPVP

SEQ ID NO: 10 - protein, 20 aa, adeno-associated virus 2
IPNYLLPKTQPELQMAWTNM

SEQ ID NO: 11 - protein, 45 aa, adeno-associated virus 4
LPNWFAVTKTRNGAGGGNKVVDDCYIPNYLLPKTQPELQMAWTNM

SEQ ID NO: 12 - protein, 71 aa, Adeno-associated virus 8
VLGRFLSQIREKLGPDHLPAGSSPTLPNWFAVTKDAVMAPAGGNKVVDECYIPNYLLPKTQPELQWAWTNM

SEQ ID NO: 13 - protein, 533 aa, adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 735 aa, adeno-associated virus 2 [sequence omitted]

SEQ ID NO: 15 - protein, 204 aa, adeno-associated virus 2
METQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

(19) United States
(12) Patent Application Publication
(10) Pub. No.: US 2016/0272687 A1
(43) Pub. Date: Sep. 22, 2016

(54) ADENO-ASSOCIATED VIRUS "X" ONCOGENE

(71) Applicant: THE BOARD OF TRUSTEES OF THE UNIVERSITY OF ARKANSA, Little Rock, AR (US)

(72) Inventor: Paul L. Hermonat, Little Rock, AR (US)

(73) Assignee: The Board of Trustees of the University of Arkansas, Little Rock, AR (US)

(21) Appl. No.: 15/035,228

(22) PCT Filed: Nov. 8, 2014
(86) PCT No.: PCT/US14/64705

§ 371 Date: May 7, 2016

Related U.S. Application Data
(60) Provisional application No. 61/901,995, filed on Nov. 8, 2013.

(51) Int. Cl. C07K 14/005; C12N 7/00

(52) U.S. Cl. CPC C07K 14/005; C12N 7/00; C12N 2750/14152; C12N 2750/14122; C12N 2750/14143

(57) ABSTRACT

A novel gene “X” of adeno-associated virus is presented, which is found to be an oncogene and to promote efficient production of recombinant AAV virus particles that may be used for human gene therapy. Since the AAV X gene appears to be an oncogene, it is desirable that it not be included in active form in recombinant AAV virus particles. Therefore A therapeutic composition comprising: a plurality of recombinant adeno-associated virus (AAV) virus particles comprising native AAV DNA and recombinant therapeutic DNA, wherein none of the AAV virus particles has an active AAV X gene is presented. Also provided are methods of expressing the X gene to improve production of recombinant AAV virus particles.

### GOVERNMENT SUPPORT

This invention was made with government support under grant R56 AI093695 awarded by the United States National Institutes of Health. The government has certain rights in the invention.

### BACKGROUND

First utilized in 1984 (1-3), adeno-associated virus (AAV) (type 2) is rapidly growing in popularity as a preferred gene therapy vector with a long transgene delivery period and high safety record (4-6). From the sequencing of adeno-associated virus type 2 (AAV2) in 1983 and the phenotypic study of AAV mutants, there have been three trans phenotypes identified within the AAV2 genome (1,7). The rep phenotype, defective for DNA replication and transcription, encodes replication/transcription factor proteins Rep78, Rep68, Rep52, and Rep40. Another trans phenotype discovered is lip (described as inf by Barrie Carter's group) (1,7) which produces viral particles of low infectivity (missing VP1). The third phenotype discovered is the cap genotype which doesn't produce any viral particles at all (encoding the major structural protein, VP3). Just recently, a fourth trans phenotype, the AAP gene, involved in virion maturation, has been identified by Jurgen Kleinschmidt (8).

Better understanding of AAV would be desirable to improve its use as a human (or nonhuman animal) gene therapy vector.

### SUMMARY

The inventor has found that adeno-associated virus type 2 (AAV2) encodes a gene we have termed “X” that has a pro-growth effect on mammalian cells in which it is active and is a likely oncogene. It also promotes AAV replication and is useful to improve efficiency and yield of production of recombinant AAV used for gene therapy.

In AAV2, the X gene is located at nucleotides 3929-4393, which is within the cap gene at nucleotides 22034410, but in a different reading frame from the three proteins encoded by CAP (VP1 at nt 2203-4410, VP2 at nt 2614-4410, and VP3 at nt 2809-4410). The native promoter for X in AAV2 is p81 at nt 3703-3813 The nucleotide numbers are from NC_001401 (AAV2).

The X protein was identified during active AAV2 replication using a polyclonal antibody against a peptide starting at amino acid 38. Reagents for the study of X were made that included (a) an AAV2 deletion mutant (dl78-91); (b) a triple nucleotide substitution mutant in which all three of the 5′ AUG-initiation products of X were destroyed with no effect on the cap coding sequence; and (c) X-positive-HEK293 cell lines. It was found that X up-regulates AAV2 DNA replication in differentiating keratinocytes (without helper virus, autonomous replication) and also in various forms of HEK293 cell assays with help from wild type adenovirus type 5 (wt Ad5) or Ad5 helper plasmid (pHelper). The strongest contribution by X was seen in increasing wt AAV2 DNA replication in keratinocytes and dl78-91 in Ad5-infected X-positive-293 cell lines (both having multi-fold effects). Mutating the X gene in pAAV-RC (pAAV-RC-3Xneg, the triple nucleotide substitution mutant mentioned above) yielded approximately a ˜33% reduction in defective recombinant AAV vector DNA replication and virion production, but a larger effect was seen when using this same X-knockout AAV helper plasmid in X-positive-293 cell lines versus normal 293 cells (multi-fold). Taken together these data strongly suggest that AAV2 X is a gene/protein involved in the AAV life cycle, particularly in increasing AAV2 DNA replication.

We also found that AAV2 X gene expression in swiss albino 3T3 cells oncogenically transforms the cells. They lose their contact inhibition. AAV2 X also increased metabolic activity of the same cells and increases their growth rate at all concentrations of fetal bovine serum supplementation in growth media. This suggests that the AAV X gene is an oncogene, and is therefore a possible health risk in human gene therapy. Incorporation of the X gene in a patient's genome could be tumorigenic. It therefore may be advisable to produce therapeutic recombinant AAV virus particles for gene therapy that do not contain an active AAV X gene.

Since the AAV X gene enhances the yield and efficiency of AAV recombinant virus production, however, it is desirable to use the X gene in recombinant AAV virus production. Accordingly, one embodiment of the invention provides a therapeutic composition comprising: a plurality of recombinant adeno-associated virus (AAV) virus particles comprising native AAV DNA and recombinant therapeutic DNA; wherein none of the AAV virus particles has an active AAV X gene.

Another embodiment provides an engineered eukaryotic host cell comprising: a chromosomally integrated X expression cassette comprising an AAV X gene under expression control of a promoter effective to express the X gene in the host cell; wherein the host cell is in vitro.

Another embodiment provides an expression system for producing recombinant AAV virus particles, the expression system comprising: (a) a eukaryotic host cell comprising a chromosomally integrated AAV X expression cassette comprising an AAV X gene under expression control of a promoter effective to express the X gene in the host cell; (b) one or more AAV helper expression cassettes collectively encoding and expressing AAV rep and cap proteins and other AAV helper proteins; and (c) an insert replication cassette encoding an insert nucleic acid flanked by inverted terminal repeats for packaging into recombinant AAV virus particles; wherein none of the AAV helper or insert expression or replication cassettes comprises an active AAV X gene.

Another embodiment provides a method of producing recombinant AAV virus particles comprising: (a) expressing AAV X gene from a chromosomally integrated X gene in a eukaryotic host cell; (b) expressing AAV rep and cap genes in the host cell; (c) expressing AAV helper genes other than X in the host cell; (d) replicating a recombinant construct comprising a recombinant gene of interest flanked by AAV inverted terminal repeats in the host cell; and (e) packaging the replicated recombinant construct into recombinant AAV virus particles.

Another embodiment provides a method of producing recombinant AAV virus particles comprising: (a) expressing AAV X gene in a eukaryotic host cell from a promoter that is not a native AAV X gene promoter and is more active in the host cell than the native AAV X gene promoter; (b) expressing AAV rep and cap genes in the host cell; (c) expressing AAV helper genes other than X, rep, and cap in the host cell; (d)

replicating a recombinant construct comprising a recombinant gene of interest flanked by AAV inverted terminal repeats in the host cell; and (e) packaging the replicated recombinant construct into recombinant AAV virus particles.

Another embodiment provides an isolated plasmid comprising AAV cap gene, wherein the plasmid does not comprise an active AAV X gene.

Another embodiment provides a eukaryotic host cell comprising: an expression cassette comprising AAV gene X under the control of a promoter, wherein the promoter is not a native AAV promoter; wherein the eukaryotic host cell is ex vivo.

Another embodiment provides an expression system for producing recombinant AAV virus particles, the expression system comprising: one or more AAV helper expression cassettes collectively encoding and expressing AAV rep and cap proteins and other AAV helper proteins; and an insert replication cassette encoding an insert nucleic acid flanked by inverted terminal repeats for replication and packaging into recombinant AAV virus particles; wherein none of the AAV helper or insert expression or replication cassettes comprises an active AAV X gene.

Another embodiment provides a method of producing recombinant AAV virus particles comprising: (a) expressing AAV rep and cap genes in a host cell; (b) expressing AAV helper genes other than X, rep, and cap in the host cell; (c) replicating a recombinant construct comprising a recombinant gene of interest flanked by AAV inverted terminal repeats in the host cell; and (d) packaging the recombinant construct into recombinant AAV virus particles; wherein the host cell does not comprise an active AAV X gene and the method therefore does not comprise expressing an active AAV X gene in the host cell.

### BRIEF DESCRIPTION OF THE DRAWINGS

FIG. 1. Identification of the AAV2 p81 promoter. HeLa cells were transduced with the ins96-0.9Neo. The marker is a 100 bp ladder. In A expression at the 3′ end of the AAV genome was studied by reverse transcriptase primer extension (RT-PE). One primer was homologous to the Neo sequences and the other at nt 3958. B shows an S1 nuclease protection assay using a antisense DNA protector which was generated also using the 3958 primer. Hence the RT-PE product in A is of the same size as the S1 nuclease product in B. The data from these two approaches agree, and definitively identify a p81 derived transcript.

FIG. 2. Elements at the 3′ end of the AAV2 genome. Shown are the lip-cap gene as a lighter gray bar. The aav2TAX gene is shown as a darker gray bar. Upstream of it is the p81 promoter identified in FIG. 5. Also shown is the position of the poly A signal and the right inverted terminal repeat (ITR).

FIG. 3: Sequences of AAV2 X. A shows the open reading frames (ORF), reading from the natural AAV2 promoters (left to right), as analyzed by NIH ORF finder software analysis of NC_001401 (AAV2, Kleinschmidt) (SEQ ID NO:1) with their names/functions indicated at the top of the figure as determined by mutational analysis (1). B shows the nucleotide (nt) sequence of the third largest ORF, called X (9), of AAV2 with its start methionines and stop codon highlighted in grey. C shows the amino acid (aa) sequence of the X (SEQ ID NO:2). D shows a series of AAV2 isolates found in Genbank which also show the X ORF.

FIG. 4: Identification of X protein. Shown is a Western blot of protein from HEK293 cells infected with Ad5 and transfected with pSM620 (wt AAV2) plus either AAV/Neo or AAV/X/Neo. The Western blot was probed with polyclonal rabbit antibodies directed against a peptide derived from aa 38-51 of AAV2 X. While polyclonal antibodies are well known for having cross-reactivity, note that a protein of approximately 18 kDa, the predicted size of X, and is seen strongly enhanced in cells transfected with AAV/X/Neo, consistent with X.

FIG. 5: Environs of the X gene and reagents for X study. A shows the region of X at the 3′ end of AAV2. Included are the 3′ end of lip-cap (1,10), the p81 promoter (9), the poly A sequence and the 3′, right, inverted terminal repeat (ITR). B shows three nt substitution mutations in X (SEQ ID NO:3) which eliminate the products from all three 5′/amino end X start methionines, but which have no effect on the cap ORF/coding sequence (residues 394-344 of CAP are shown, SEQ ID NO:4). C shows the analysis of twelve 293 cell clones generated by transfection of pCI/X/Neo, and then G418 selected. The left scale shows the copy number of X found by Q-RT-PCR with clone D as the “1X” reference clone. 293-X-B and 293-X-K, having the highest copy numbers of X were chosen for further study.

FIG. 6: X enhances AAV2 autonomous DNA replication in skin rafts. A shows the structure of the AAV vector plasmids used. B shows the structure of the experiment analyzing X gene function in the skin raft (stratified squamous epithelium, autonomous AAV2 replication). Note that the plasmid is transfected before infection of the keratinocytes with wt AAV. This is done so as to allow the transfected gene to be expressed during the early phase of wt AAV replication. C shows the resulting Southern blot of DNA after probing the membrane with 32P-cap sequences (but not including X sequences). D shows a quantification of five such experiments. Note that AAV2 DNA replication is enhanced 6 fold. E is an ethidium-bromide stained agarose gel of a reverse transcriptase cDNA-PCR amplification of mRNA with primer sets for amplifying both TFIIIB and rep mRNAs. Panel E shows that X enhances AAV2 rep mRNA expression relative to housekeeping TFIIB gene expression. These data are fully consistent with the higher DNA replication found in C. F is a Southern blot of equal total DNAs from the cells with the indicated plasmid transfection. F shows dosage dependent effect of adding X. Note that the larger the amount of AAV/X/Neo transfected the higher the level of AAV2 DNA replication.

FIG. 7: Deletion of X gives lower DNA replication of AAV2. A shows the structure of AAV2 deletion mutants dl63-78 and dl78-91, with wild type (wt) AAV2 shown at the top, including Pst I restriction sites. B shows a Pst I, Bgl II dual digestion of dl63-78 and dl78-91. C shows a Southern blot analysis comparison of dl63-78 and dl78-91 DNA replication in Ad5-infected 293 cells, probed with 32P-rep DNA, and densitometrically quantitated in D. Note that dl63-78 replicates approximately 2.5 fold higher than the dl78-91. E shows a comparison of dl78-91 DNA replication upon co-transfection with either AAV/Neo or AAV/X/Neo into Ad5-infected 293 cells. F shows a Southern blot analysis comparison of dl78-91 DNA replication in Ad5-infected unaltered 293 cells, 293-X-B, and 293-X-K, probed with 32P-rep DNA. An analysis of the level of copy numbers of X in these cells is shown in FIG. 5 C. Note that dl78-91 replicates to higher levels in the 293 cells which contain the X gene (complementation) compared to unaltered 293 cells without X.

FIG. 8: pSM620-3Xneg, without X, displays weaker DNA replication in Ad5-infected 293 cells. A shows a Pst I restriction digestion analysis of wt pSM620 and pSM620-3Xneg (X−). B shows the Southern blot of DNA replication, using a ³²P-rep probe, of pSM620 and pSM620-3Xneg relative to each other in Ad5-infected 293 cells. Note that pSM620 replicated to a slightly higher level than pSM620-3Xneg. C shows a “2^{nd} plate analysis” where equal aliquots of virus stock from plates identical to those of B were heated to 56° C. (to kill Ad5), and then used to infect a second plate of Ad5-infected 293 cells. Shown is the Southern blot of DNA replication, using P32-rep probe, of pSM620 and pSM620-3Xneg replication resulting from first-plate-generated virus infection. D shows a Southern blot analysis comparison of pSM620-3Xneg replication in Ad5-infected unaltered 293 cells, 293-X-B, and 293-X-K, probed with ³²P-rep DNA. Note that pSM620-3Xneg replicates to higher levels in the 293 cells which contain the X gene (complementation) compared to 293 cells without X. E shows another “2^{nd} plate analysis” where equal aliquots of virus stock from plates identical to those of D were heated to 56° C. (to kill Ad5), and then used to infect a second plate of Ad5-infected 293 (normal) cells. Shown is the Southern blot of DNA replication, using ³²P-rep probe, of pSM620-3Xneg replication from resulting first plate generated virus infection. Note that, pSM620-3Xneg replicated to higher levels in the 2^{nd} plate due to higher levels of virus produced in the first plate.

FIG. 9: Recombinant defective (r)AAV DNA replication and virion production are lower without X. A shows the Southern blot analysis of rAAV/eGFP DNA replication, using ³²P-eGFP probe, resulting from the standard 293 cell triple transfection procedure (pAAV/eGFP, pHelper, pAAV-RC) except comparing the usage of either wt AAV-RC or pAAV-RC-3Xneg. Note that use of pAAV-RC resulted in slightly higher pAAV/eGFP DNA replication levels than when using pAAV-RC-3Xneg. B shows a Southern blot analysis of DNAse-I-resistant virion DNA (encapsidated genomes). Again note that the use of wt pAAV-RC resulted in slightly higher rAAV/eGFP virion levels. C shows a Southern blot (³²P-eGFP probe), which compares the use of pAAV-RC-3Xneg, along with pAAV/eGFP and pHelp, to replicate AAV/eGFP DNA in unaltered 293, versus 293-X-B and 293-X-K cells, both of which contain the X gene. Note that higher DNA replication levels of AAV/eGFP take place in the X-positive 293-X-B and 293-X-K cells than normal 293 cells. D shows a Southern blot analysis of DNAse-I-resistant virion DNA (encapsidated genomes). Again note that the use of 293-X-B and 293-X-K cells, having the X gene, resulted in higher rAAV/eGFP virion levels. E shows an analysis of eGFP expression/virion infectivity in which AAV/eGFP virus, equalized for comparable titers from quantitative densitometric analysis of the virion DNA Southern blot in panel D was used to infect normal 293 cells and analyzed for eGFP expression at two days post-infection. Note that equal eGFP expression can be seen across all three cell infections indicating that the use of pAAV-RC-3Xneg with the 293-X-positive cell lines gave virus with comparable infectivity to the standard pAAV-RC/wt 293 cell production scheme. F show a white light picture of the same field depicted in E as a control for cell viability.

FIG. 10. Focus formation/loss of contact inhibition of 3T3-swiss albino mouse fibroblasts by AAV2 X protein using virus infection. The two swiss albino cell lines, SA3T3-XneoV and SA3T3-neoV, were seeded onto 6 cm plates and allowed to grow for two weeks post confluence, then methylene blue stained. Panel A shows a photograph of the plates as indicated. Panel B shows a quantification of the foci. Panel C shows PCR analysis that the SA3T3-XneoV cells contain AAV2 X DNA. Note that SA3T3-XneoV cells displayed significantly more foci, loss of contact inhibition, than the SA3T3-neoV cells. Panels D and E are photomicrographs of the cells from plates of Panel A. Representative fields at 100× magnification. Note that the swiss albino 3T3-AAV/Neo cells (bottom) appear regular and display contact inhibition. In contrast the AAV/“X”/Neo cells (top) display loss of contact inhibition with cell stacking and much higher density.

FIG. 11. Serum-dependent growth of 3T3-swiss albino fibroblasts expressing AAV2 X protein. The same SA3T3-XneoV and SA3T3-neoV cells from FIG. 2 (5λ10″) were assayed for serum dependence. Panel A shows the plates fed with 10%, 1%, and 0.5% FBS. Panel B shows a magnification of the plates fed with 1% and 0.5% FBS. Note that the same SA3T3-XneoV cells (left) always grew more extensively for any given FBS concentration than the SA3T3-neoV cells.

FIG. 12. Effects of AAV2 X on invasion by 3T3-swiss albino mouse fibroblasts. The same SA3T3-XneoV and SA3T3-neoV cells from FIG. 10 were tested for growth in soft agar. However, while no colony growth was seen by either cell type, as can be seen, invasion from, out of the agar and onto the bottom of the plate, was found to be much more extensive for the SA3T3-XneoV cells, than the SA3T3-neoV cells. Moreover, the cell density of the invading cells was much higher by the SA3T3-XneoV cells. These data further confirm the phenotype of X as a pro-growth gene.

FIG. 13. Loss of contact inhibition of 3T3-swiss albino mouse fibroblasts by AAV2 X using plasmid transfection. This experiment is similar to FIG. 2, but involves cells generated by plasmid transfection instead of virus infection. Swiss albino 3T3 cells were calcium phosphate transfected with pC|-Neo (negative ctrl), pCl-X-Neo, or L67N (positive ctrl)(5 μg each), G418 selected, to give the bulk cell lines SA3T3-Xneo, SA3T3-neo, SA3T3-L67neo. Panel A shows the plates as indicated. Panel B shows a visual quantification of the foci. Note that SA3T3-neo cells gave no foci, whereas both the SA3T3-Xneo and SA3T3-L67neo cells did generate foci. Panel C shows that the SA3T3-Xneo cells contain X DNA.

FIG. 14. X DNA from helper plasmids is packaged into AAV virus particles. Shown is the analysis of virion DNA, as indicated, by PCR amplification of full length of the X ORF DNA. “AAV2-CAG-GFP virus DNA” is DNase-resistant DNA from cesium chloride gradient purified virus (5×10¹⁰ virus) purchased from VECTOR BIOLABS (Cat No 7072) used as template. “AAV2/CMV-GFP virus DNA” is DNase-resistant DNA from one of our own virus stocks as template. pCl-X-neo plasmid is a positive control and dH₂O is a negative control. Note that both virus stocks contained full length X ORF DNA.

FIG. 15. AAV2 “X” protein has homology to MED19, HTLV2 TAX, and BAF53A/ACTL6. Shown are results from NCBI Protein Blast analysis. Panel A shows a bar graph comparison of the homology of HTLV2 Tax, MED19, HPV 68 E6, and ACTL6 against the AAV2 X aa sequences (155aa). Panels B and C show amino acid homologies of MED19 (SEQ ID NO:6) and HPV 68 E6 (SEQ ID NO:5) with AAV2 X (SEQ ID NO:2) by NCBI Protein Blast analysis. Note that MED19 is closest in size to X (181 versus 155 aa) and therefore might serve as a better model for X.

FIG. 16. The AAV6 genome showing Xa and Xb. Shown in panel A are the ORFs of AAV6 by NIH ORF finder derived from the Genbank AF028704 (SEQ ID NO:7) but with the AAV X region replaced with sequences from EU368909. Note that there are two open reading frames, Xa and Xb, present in the position occupied by AAV2 X Panel B shows the DNA and amino acid (SEQ ID NO:8) sequences of Xa. Panel C shows the DNA and amino acid (SEQ ID NO:9) sequences of Xb.

FIG. 17. Homologies between AAV2 X and AAV6 Xa and Xb. Shown in panel A is a more detailed caricature of the X ORFs of AAV6 by NIH ORF finder derived from the Genbank AF028704 plus EU368909. In panel B is shown an NCBI Protein BLAST analysis of the artificially fused AAV6 Xa-Xb (SEQ ID NOS:7 and 8) amino acid sequence with that of AAV2 X (SEQ ID NO:2). Note that the homology of the two X sequences extends the length of fused AAV6 Xa-Xb.

FIG. 18. AAV2 X helps AAV6 rep and cap In AAV2/6.eGFP production. Here we demonstrate that AAV2 X also helps an AAV6-based rep/cap system. We used pRepCap6 which has both the AAV6 rep and cap genes. 293-X-B and 293-X-K are cell lines which contain the X gene. Panel A shows a Southern blot of pAAV/eGFP DNA replication probed with ³²P-eGFP DNA. Panel B is a densitometric quantification of panel A. Note that in the presence of AAV2 X, included In 293-X-B and 293-X-K, the level of AAV/eGFP DNA replication was significantly higher compared to 293 cells. Panel C shows a dot blot of DNaseI-resistant pAAV/eGFP virion DNA probed with ³²P-eGFP DNA. Panel D is a densitometric quantification of panel C. Note that in the presence of AAV2 X, included In 293-X-B and 293-X-K, the level of AAV/eGFP virion DNA was significantly higher compared to 293 cells.

FIG. 19. AAV2 X helps rAAV/Foxp3 DNA and virion production driven by AAV6 rep/cap. Here we demonstrate that AAV2 X also helps an AAV6-based rep/cap system. We used pRepCap6 which has both the AAV6 rep and cap genes. 293-X-B and 293-X-K are cell lines which contain the X gene. Panel A shows a Southern blot of pAAV/Foxp3 DNA replication (probed with ³²P-Foxp3 DNA. Panel B is a densitometric quantification of A. Note that in the presence of AAV2 X, included In 293-X-B and 293-X-K, the level of AAV/Foxp3 DNA replication was significantly higher compared to 293 cells. Panel C shows a dot blot of DNaseI-resistant pAAV/Foxp3 virion DNA probed with ³²P-Foxp3 DNA. Panel D is a densitometric quantification of panel C. Note that in the presence of AAV2 X, included In 293-X-B and 293-X-K, the level of AAV/Foxp3 virion DNA was significantly higher compared to 293 cells.

FIG. 20. Homology between Xs and Rep78s. Shown in panels A, B, C, and D are NCBI Protein Blast homology analyses between AAV2 X and AAV type 2, 4, 8, and Go.1 Rep78/NS1 proteins. Note that for most Rep78s the homology with X lies in a region from 100-200 amino acids. These homologies suggest that Rep78 DNA sequences may have exchanged material with the 3′ end of the AAV genomes. AAV8 may be the most likely source due to its longest length and homology. However, for Go.1, related to AAV5, the homology resides at the extreme carboxy-terminus of Rep78. AAV2 Rep78 is SEQ ID NO:10; AAV4 Rep78 is SEQ ID NO:11; and AAV8 Rep 78 is SEQ ID NO:12.

### DETAILED DESCRIPTION

One embodiment of the invention provides a therapeutic composition comprising: a plurality of recombinant adeno-associated virus (AAV) virus particles comprising native AAV DNA and recombinant therapeutic DNA; wherein none of the AAV virus particles has an active AAV X gene. In Example 3 below we show that AAV X has oncogenic properties. Thus, it may be desirable for therapeutic AAV particles to not contain an active X gene. X is found in a portion of the cap gene, which is a necessary in AAV helper plasmids. Some of the helper DNA gets incorporated into virus particles. If active X is only present as chromosomally integrated gene in the host cell producing virus, and not in plasmid or virus DNA, no virus particles will have incorporated active X genes. The other way to insure no active X genes are present in virus particles is to disable X in the helper plasmid by mutation.

The “helper” genes are genes not provided on an engineered AAV that help the AAV to replicate or help virus production. Some helper genes are native to the host cell; they are host cell genes necessary or in some cases merely helpful for virus replication or virus particle production. Other helper genes are provided by a plasmid or other vector that is transformed or otherwise engineered to be in the host cell. The helper genes can also be foreign or engineered genes that are integrated into the host cell chromosome. A “helper plasmid” is a plasmid that contains at least one helper gene. A “helper expression cassette,” is an expression cassette that contains at least one helper gene and that is not part of the engineered AAV genome. An expression cassette as used herein, refers to any gene under control of a promoter and any other elements that may regulate or control its expression. The expression cassette may be or include a native gene and promoter of a host cell chromosome, or a gene or promoter not native to the host cell, and may be on a chromosome or plasmid and be engineered or not.

Some of the AAV helper genes are listed in Table 1. Other helper genes exist and not every helper gene listed is necessary or included in every host cell producing AAV.

TABLE 1
Selected AAV helper genes

| Source | Gene | Function |
|---|---|---|
| Adenovirus | E1A | Oncogene |
| | E1B | Oncogene |
| | E2A | ss DNA binding |
| | E4orf6 | Oncogene and other |
| | | functions |
| | VA1 | Small RNA inhibitory |
| Human | E1 | Helicase |
| papilloma virus | E2 | DNA binding transcription |
| | | factor |
| | E6 | Oncogene |
| Herpes | ICP0, CIP4, and ICP22 | Transcription factors |
| simplex virus | UL5, UL8, UL52 | Make the HSV primase |
| | UL30, UL42 | HSV DNA polymerase |
| | UL29 | ss DNA binding |
| | LANA | Binds and regulates |
| | | transcription factors. |
| Mammalian | DNA pol delta | DNA polymerase subunit |
| host cell | PCNA | DNA polymerase cofactor |
| | RFC | Nucleotide synthesis |
| | RPA | ss DNA binding |
| | MCM5 | Chromatin binding |

AAV genes may also be helper genes, including the genes encoding the proteins X (SEQ ID NO:2), rep, cap (SEQ ID NO:13), lip (SEQ ID NO:14), and AAP (SEQ ID NO:14). The coding sequence for X is nucleotides 3929-4393 of SEQ ID NO:1. The coding sequence for cap is nucleotides 2809-4410 of SEQ ID NO:1. The coding sequence for lip is nucleotides 2203-4410 of SEQ ID NO:1. The coding sequence for AAP is nucleotides 2729-3343 of SEQ ID NO:1. The rep gene spans nucleotides 321 to 2252 of SEQ ID NO:1 and four variants are expressed based on alternative splicing and translation. Rep 68 is encoded by nucleotides 321-1906 joined to 2228-2252 of SEQ ID NO:1. Rep 78 is encoded by nucleotides 321-2186 of SEQ ID NO:1. Rep 40 is encoded by nucleotides 993-1906 joined to 2228-2252 of SEQ ID NO:1. Rep 52 is encoded by nucleotides 993-2186 of SEQ ID NO:1.

Another embodiment provides an engineered eukaryotic host cell comprising: a chromosomally integrated X expression cassette comprising an AAV X gene under expression control of a promoter effective to express the X gene in the host cell; wherein the host cell is in vitro. This is useful to produce recombinant AAV particles that have no active X gene and/or to enhance production of recombinant AAV particles.

In a specific embodiment, the host cell is a HEK293 derivative. The term “HEK293 derivative” is intended to include HEK293 and engineered HEK293, such as by incorporation of a chromosomally integrated copy or copies of X or a plasmid copy or copies of X.

In specific embodiments where a chromosomally integrated X gene or plasmid X gene is present, the X gene may be under the control of a promoter that is not a native AAV X gene promoter. The native AAV2 X gene promoter is p81, as disclosed in Example 1 below. Other AAV stains have their own native X gene promoters, which may correspond in location and sequence to p81 or may be different promoters. Other native AAV X gene promoters may also be present but not yet characterized in AAV genomes. The term “native AAV X gene promoter” includes any promoter in an AAV strain that in nature drives expression of an AAV X gene.

In a particular embodiment, the promoter effective to express the X gene in a host cell is cytomegalovirus (CMV) immediate early promoter (CMV promoter). Other promoters suitable for use to express X in a eukaryotic host cell are known to persons of ordinary skill in the art.

In particular embodiments, the promoter effective to express the X gene in the host cell gives higher expression in the host cell than the native X gene promoter. That is, if the promoter is linked in an expression construct to a reporter gene it gives higher expression of the reporter gene in the host cell than an otherwise identical expression construct with the native X gene promoter linked to the reporter gene in the same host cell type.

Another embodiment provides an expression system for producing recombinant AAV virus particles, the expression system comprising: (a) a eukaryotic host cell comprising a chromosomally integrated AAV X expression cassette comprising an AAV X gene under expression control of a promoter effective to express the X gene in the host cell; (b) one or more AAV helper expression cassettes collectively encoding and expressing AAV rep and cap proteins and other AAV helper proteins; and (c) an insert replication cassette encoding an insert nucleic acid flanked by inverted terminal repeats for packaging into recombinant AAV virus particles; wherein none of the AAV helper or insert expression or replication cassettes comprises an active AAV X gene.

In specific embodiments, the other AAV helper proteins and genes may comprise lip or cap or both. In other specific embodiments, the AAV helper proteins and genes comprise genes or proteins of one or more other viruses, such as adenovirus, human papilloma virus, and herepes simplex virus, e.g., those listed in Table 1.

In other specific embodiments, the other AAV helper genes or proteins may only comprise native genes or proteins of the host cell, i.e., mammalian genes or proteins.

In a particular embodiment, the chromosomally integrated X expression cassette is not a part of a full active chromosomally integrated cap gene.

Another embodiment provides a method of producing recombinant AAV virus particles comprising: (a) expressing AAV X gene from a chromosomally integrated X gene in a eukaryotic host cell; (b) expressing AAV rep and cap genes in the host cell; (c) expressing AAV helper genes other than X, rep, and cap in the host cell; (d) replicating a recombinant construct comprising a recombinant gene of interest flanked by AAV inverted terminal repeats in the host cell; and (e) packaging the replicated recombinant construct into recombinant AAV virus particles.

The method may further comprise purifying the recombinant AAV virus particles.

Another embodiment provides a method of producing recombinant AAV virus particles comprising: (a) expressing AAV rep and cap genes in a host cell; (b) expressing AAV helper genes other than X, rep, and cap in the host cell; (c) replicating a recombinant construct comprising a recombinant gene of interest flanked by AAV inverted terminal repeats in the host cell; and (d) packaging the recombinant construct into recombinant AAV virus particles; wherein the host cell does not comprise an active AAV X gene and the method therefore does not comprise expressing an active AAV X gene in the host cell.

In specific embodiments of the methods described herein, the host cell is in vitro. In other embodiments, it is in vivo.

In specific embodiments, the host cell is a HEK293 derivative.

### EXAMPLES

### Example 1

### Identification of p81 Promoter in Adeno-Associated Virus Type 2 (AAV2) p81 Promoter and Hypothetical Open Reading Frame “X”

### p81 Promoter

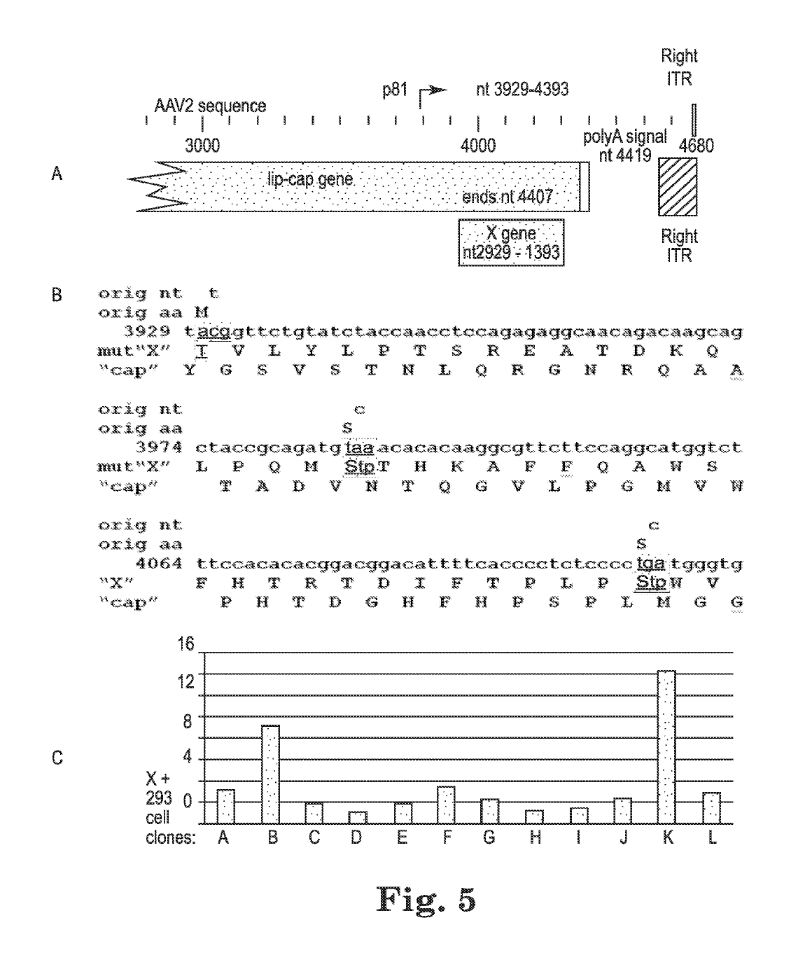

We identified a previously unknown promoter in the Lip-Cap gene in AAV2 at nt 3793-3813. HeLa cells were transduced with the Ins96-0.9Neo AAV. Ins96 is a genetically and phenotypically wild-type AAV genome (Hermonat, P L et al., 1984, Proc. Natl. Acad. Sci. USA 81:6466-6470). To create Ins96-0.9Neo, the 960-base neo gene was ligated into the BglII sit at nt 4483 if Ins96. Expression at the 3′ end of the AAV gene was studied by reverse transcriptase primer extension (RT-PE). One primer was homologous to the Neo sequences and the other to a sequence ending at nt 3958. FIG. 1A shows that primer extension with the nt 3958 primer produced an extension product approximately 100 nt long. FIG. 5B shows an S1 nuclease protection assay using an antisense DNA protector which was generated using the 3958 primer. The S1-protected product in B is the same size as the transcript in FIG. 1A. These data from the two approaches agree and identify a transcript derived from the p81 promoter at nt 3793-3813.

### X Open Reading Frame

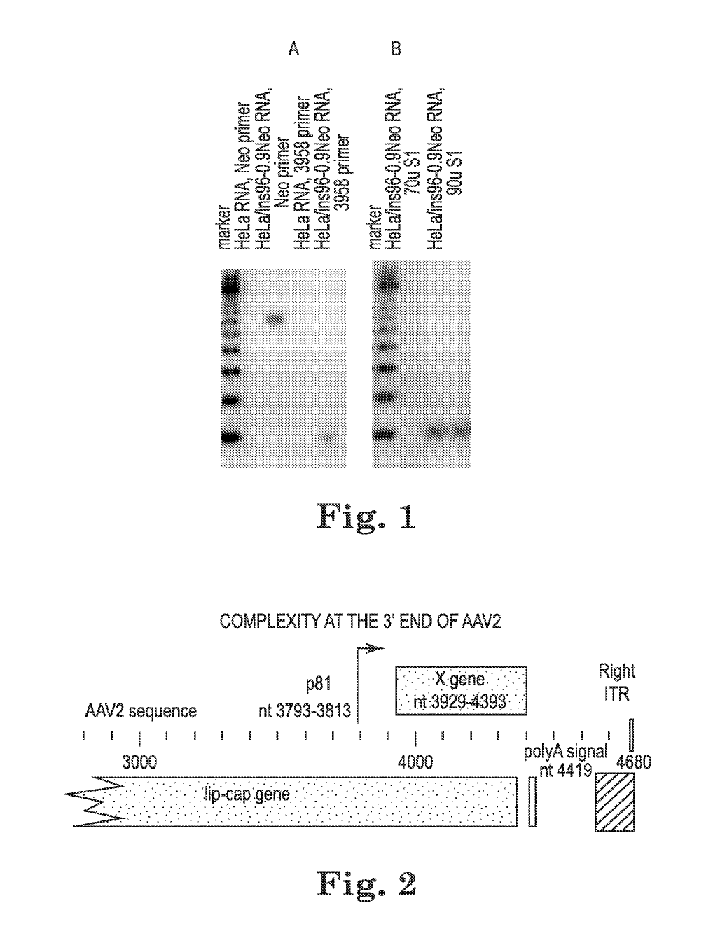

Downstream of this promoter is an open reading frame we termed “X” at nt 3929-4393 of AAV2, as shown in FIG. 2. Two other alternative ATG start codons are also shown.

### Example 2

### AAV “X” Promotes AAV Replication and Efficient Generation of Recombinant AAV

In this Example, we investigated whether the open reading frame “X” described in Example 1 is actually expressed as a protein, and what the function of the protein might be.

### Results.

### Computer Analysis and Generation of X Reagents.

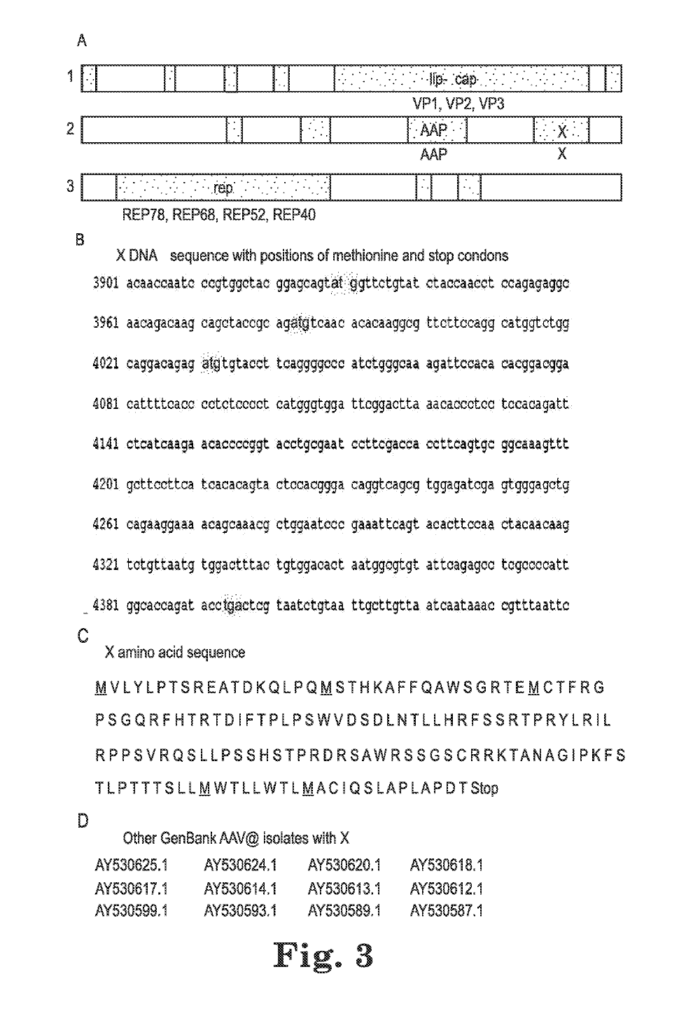

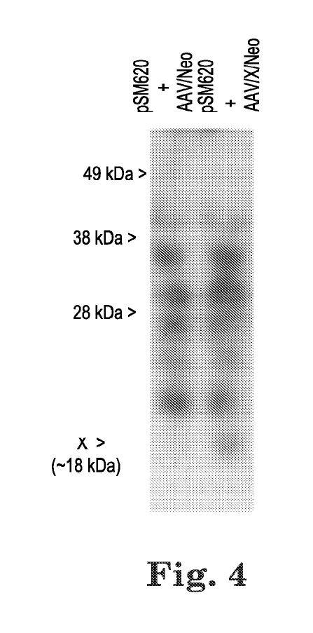

X is a rather significant ORF of 465 base pairs, 155 amino acids. FIG. 3A, shows a cartoon of the AAV2 genome and includes the relative position of genes/open reading frames (ORF). FIG. 3B shows the DNA sequence of the AAV2 X ORF derived from NCBI Reference Sequence NC_001401.2, and FIG. 3C shows the corresponding amino acid sequence derived from the X ORF. FIG. 3D shows other sequences of AAV2 isolates that also contain the X ORF. We analyzed whether we might be able to identify an actual X protein. FIG. 4 shows a western blot of protein from Ad5-infected 293 cells, with pSM620 (wt AAV2) co-transfected with AAV/Neo or AAV/X/Neo, which identifies an enhanced protein band at the correct size (X is ˜18 kDa) only when AAV/X/Neo is present.

Given this evidence that X is an actual protein, to study X we generated multiple types of constructs (FIG. 5). The vicinity of X within the AAV2 genome is shown in FIG. 5A. One mutant, dl78-91, based on pSM620 (wild type AAV2), has a large deletion of X (hitting cap as well) which also eliminates the previously identified p81 promoter driving expression of X (9). A second mutant (FIG. 5B) is a triple knockout of the X ORF without any effect on the coding of the cap gene. That is, VP1, VP2 and VP3 remain unaltered, while all products from the three 5′ start methionines of the X ORF are eliminated. This triple mutant was inserted into both pAAV-RC and pSM620 to give pAAV-RC-3Xneg and pSM629-3Xneg, respectively. Finally, a series of 293 cell lines were generated carrying the X gene by transfecting with pCI/X/Neo and then carrying out G418 selection. A number of these 293-X-Neo resistant cell lines were analyzed by Q-PCR to determine the copy number of X which they had within (FIG. 5C). 293-X clones B and K were chosen for further study as they had the highest X copy number among the 12 clones analyzed.

### X Contributes to Autonomous AAV2 Replication in Skin Rafts.

X may be involved in wild type AAV2's replication in natural host tissue, stratified squamous epithelium, such as that found in the nasopharynx or genital tract, known to harbor AAV2. Additionally AAV2 is known to autonomously replicate in differentiating skin cells (11-14). We therefore utilized the organotypic epithelial raft culture system (skin raft) to analyze effects of X on AAV2 autonomous DNA replication. Primary human foreskin keratinocytes (PHFK) were transfected with X-expressing plasmid (or control Neo only plasmid) as shown in FIGS. 6A and B, and then these cells were subsequently infected with wild type AAV2. The next day these cells were used to generate a skin raft as shown in FIG. 6B. After day 5 of keratinocyte stratification (skin development) the skin rafts were harvested and analyzed for both DNA replication and rep gene RNA expression. FIG. 6C shows a ³²P-cap DNA probed Southern blot of a representative gel (of three total). As can be seen the level of monomer duplex (md) AAV2 DNA (4.7 kb) is approximately six fold higher in the presence of X plasmid transfection when analyzed by densitometric quantification of the autoradiograph shown in FIG. 6D. A reverse transcriptase polymerase chain reaction (RT-PCR) analysis of rep RNA expression was done and FIG. 6E shows that the ratio of rep to TFIIB housekeeping control gene was highest in the presence of X plasmid transfection, consistent with the higher AAV2 DNA replication. We further analyzed the effects of X on AAV2 replication in a similar type of experiment to that of FIG. 6C, shown in FIG. 6F, but with an increasing transfection of the X expression plasmid, as indicated. It was found that increasing doses of X plasmid resulted in corresponding increasing levels of autonomous AAV2 DNA replication. This analysis confirms the importance of X in autonomous wild type AAV2 DNA replication.

X Contributes to dl78-91 DNA Replication in HEK-293 Cells.

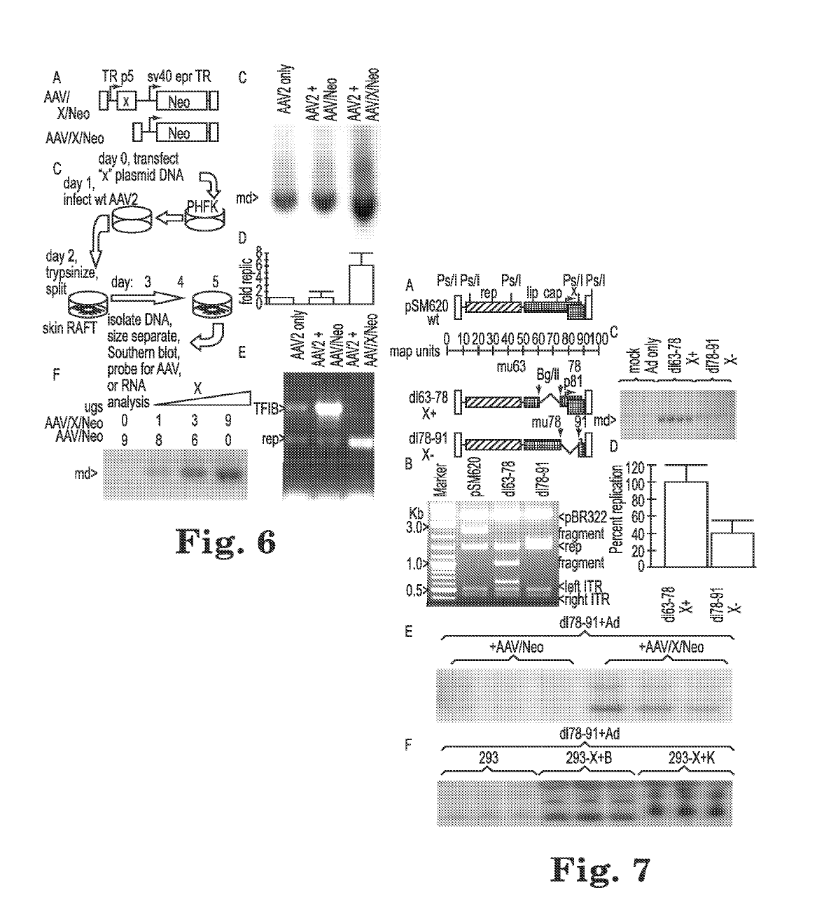

While not mimicking any normal primary cells as the primary keratinocytes do, we next tested the involvement of X in the AAV2 life cycle in HEK-293 cells using the somewhat similar assay as to that in FIG. 5. Ad5 infected (moi of 10)-293 cells were transfected with a deletion mutant (dl) dl63-78 or dl78-91 plasmid. The structure of these two mutants is show in FIG. 7A and an analysis of their structures by Pst I restriction digestion is shown in FIG. 7B. The transfected 293 cells were harvested at two days post-Ad5 infection and total cellular DNA analyzed for AAV2 DNA replication by Southern blot using 32P-rep probe, shown in FIG. 7C, and a densitometric quantification of the results is shown in FIG. 7D. As can be seen dl63-78, with an intact X gene, was able to replicate at a 2.5 fold higher level than dl78-91, again suggesting that X is involved in AAV2 DNA replication in 293 cells, as it was found in differentiating primary keratinocytes. We next tried to complement the defective phenotype of dl78-91. Ad5 infected (moi of 10)-293 cells were transfected with dl78-91 plasmid plus X expressing plasmid, or control Neo-only plasmid. DL78-91 is a deletion mutation of p81-X, as shown in FIG. 5B, and as such it is rep+, can replicate its DNA, but can't make virus as it is also cap-. The transfected 293 cells harvested at two days post-Ad5 infection and analyzed for both DNA replication. FIG. 7A shows a ³²P-rep DNA probed Southern blot, and as can be seen the level of monomer duplex (md) AAV2 dl78-91 DNA (4.1 kb) is several times higher in the presence of X-expressing plasmid, and consistent with the earlier data (FIG. 7E). We then used the X-positive 293 cells (293-X-B and 293-X-K; see FIG. 7C) and used these cells to confirm complementation of dl78-91 by X to give higher DNA replication. DL78-91 plasmid was transfected into equivalent plates (70% confluent) of unaltered 293, 293-X-B, and 293-X-K, all of which had all been infected with Ad5 (moi 10). In the resulting Southern blot, it is shown that dl78-91 reached a higher level of DNA replication, indicating the beneficial effects of X expression (FIG. 7F).

X Contributes to AAV2 (pSM620) DNA Replication in 293 Cells.

To determine the effect of X within the context of the complete AAV2 genome we compared fully wild type pSM620 to pSM620-3Xneg. Ad5-infected-(moi of 10)-293 cells were transfected with pSM620 to pSM620-3Xneg plasmid and the DNA of the transfected 293 cells harvested at two days post-Ad5 infection and analyzed for DNA replication, and equivalent plates were used to compare virion production. FIG. 8B shows a ³²P-cap DNA probed Southern blot of DNA replication, and as can be seen the level of monomer duplex (md) wt AAV2 (4.7 kb) of pSM620 was ˜33% higher than the level of AAV2-3Xneg. Equal aliquots (300 μl) of resulting virus stock were heated to 56° C. for 30 minutes (heat kill Ad5) and used to infect a second plate of Ad5-infected HEK293 cells. FIG. 8C shows a ³²P-rep (1.5 kb Pst I fragment) DNA probed Southern blot of the 2^{nd} plate DNA replication, and as can be seen the level of monomer duplex (md) wt AAV2 (4.7 kb) of pSM620 was ˜66% higher than the level of AAV2-3Xneg replication. This is consistent with an accumulative compounding of the weaker replication of pSM620-3Xneg in 2 rounds of replication.

We again used the X-positive 293 cells (293-X-B and 293-X-K) to observe if there was any form of complementation of pSM620-3Xneg during DNA replication. pSM620-3Xneg plasmid was transfected into equivalent plates (70% confluent) of unaltered 293, 293-X-B, and 293-X-K, all of which were infected with Ad5 (moi 10). In the resulting Southern blot (FIG. 8D) notice that pSM620-3Xneg reached a higher level of DNA replication in the 293-X-B and 293-X-K cells than in the 293 cells, again verifying the contribution of X to AAV2 DNA replication. Equal aliquots (300 μl) of resulting virus stock were heated to 56° C. for 30 minutes (heat kill Ad5) and used to infect a second plate of Ad5-infected HEK293 cells. FIG. 8E shows a ³²P-rep DNA probed Southern blot of the 2^{nd} plate DNA replication, and as can be seen the level of monomer duplex (md) wt AAV2-3Xneg (4.7 kb) from pSM620-3Xneg was ˜66% higher than the level of AAV2-3Xneg replication. Thus in both a head-to-head comparison of wt and 3Xneg versions of AAV2 and in 3Xneg in 293 versus 293-X positive cell lines the lack of X showed as a lower DNA replication level and production of AAV2 virus (2^{nd} plate analysis).

X Contributes to rAAV/eGFP DNA Replication and Virion Production in 293 Cells.

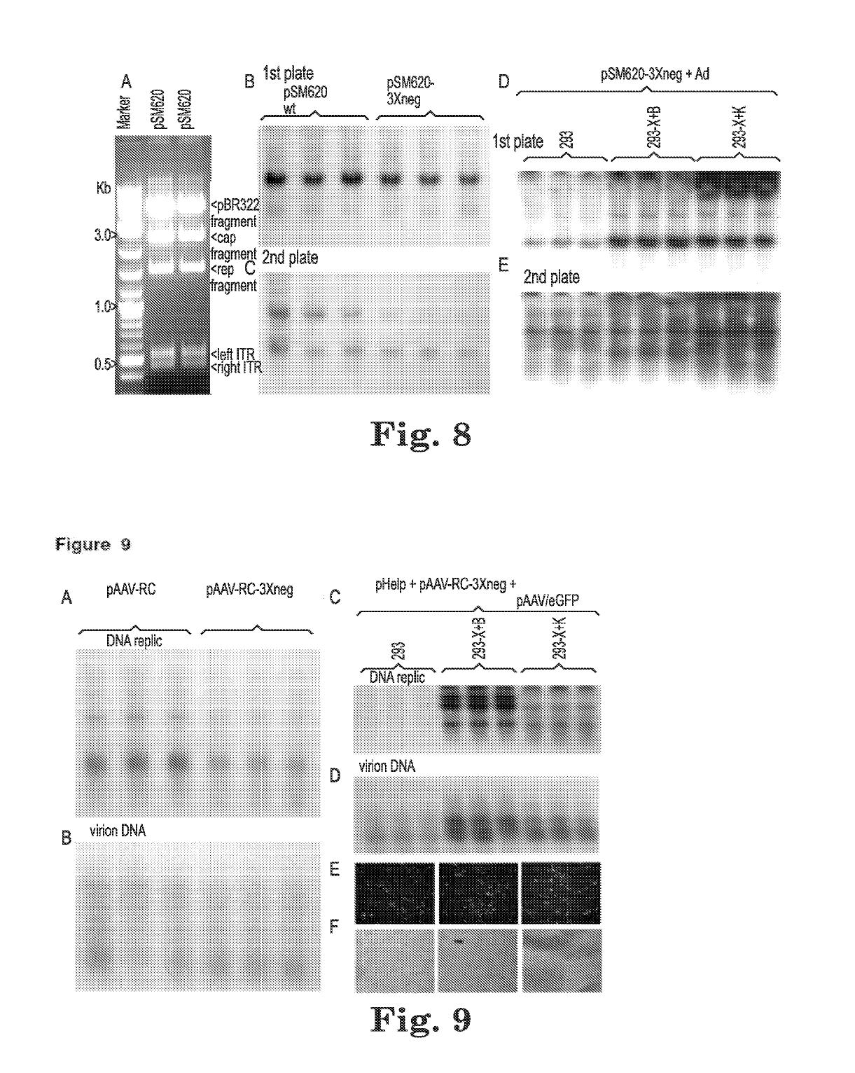

While these analyses of wild type AAV2 autonomous replication in skin and in HEK 293 cells is critically important to understand the effects of X within the normal AAV2 viral life cycle, most researchers want to known about X's effects on recombinant (r)AAV2 DNA production, as AAV-based gene delivery is now a growing industry. To determine the effect of X on rAAV production the rAAV2/eGFP virus stocks were produced by the triple transfection of pAAV/eGFP, pHelper, and pAAV-RC, with the exception that where indicated pAAV-RC3Xneg was used in place of pAAV-RC. Seventy percent confluent 293 cells were transfected with those three plasmids, including the trade off of either pAAV-RC or pAAV-RC3X and analyzed for DNA replication, and equivalent plates were used to compare virion production. FIG. 9A shows a ³²P-eGFP DNA probed Southern blot of DNA replication, and as can be seen the level of monomer duplex (md) wt AAV2/eGFP (2.0 kb) was 50% higher using wt pAAV-RC than with pAAV-RC-3Xneg. Equal aliquots (300 μl) of resulting virus stock were then analyzed for DNase I-resistant encapsidated DNA (virion DNA). FIG. 9B shows a ³²P-eGFP DNA probed Southern blot of the virion DNA which was also similarly 50% higher as was the level of DNA replication.

We again used the X-positive 293 cells (293-X-B and 293-X-K) to observe if there was any form of complementation of pAAV-RC-3Xneg during rAAV DNA replication and virion production. rAAV2/eGFP virus stocks were produced by the triple transfection of pAAV/eGFP, pHelper, and pAAV-RC-3Xneg, into the 293, 293-X-B and 293-X-K cells (70% confluent). The resulting Southern blot analysis (FIG. 9C) shows that pAAV/eGFP DNA replication levels were 4.2 fold and 2.3 fold higher in 293-X-B and 293-X-K cells, respectively, than in the unaltered 293 cells. Yet again this verifies the contribution of X to rAAV/eGFP DNA replication. Equal aliquots (300 μl) of resulting virus stock were then analyzed for DNase I-resistant encapsidated DNA (virion DNA). FIG. 9D shows a ³²P-eGFP DNA probed Southern blot of the virion DNA were 3.6 fold and 2.6 fold higher in 293-X-B and 293-X-K cells, respectively, than in virus stock from the unaltered 293 cells.

### Discussion

This Example demonstrates that AAV2 X protein is expressed and that X increases AAV2 autonomous DNA replication (no helper) in differentiating keratinocytes, its natural host tissue, in AAV2 DNA replication in Ad5-infected 293 cells, and rAAV2/eGFP replication/virion production in HEK 293 cells with complementation by pHelper and pAAV-RC plasmids.

This study demonstrates that the AAV2 X gene has an effect on AAV2 biology in two different tissue culture systems (primary keratinocytes and various forms of HEK293 cells), and the replication of both the full length AAV2 genome and fully defective rAAV2/eGFP recombinant. While not commonly used for AAV study, AAV2 is able to productively replicate, without the presence of helper virus, in the skin raft culture system (11-14), and in this system augmentation of X expression by plasmid transfection gave rise six fold higher AAV2 DNA replication. Additionally, we utilized one of the standard systems for production of rAAV2 which includes the use of pHelper (containing the Adenovirus helper genes) and pAAV-RC (containing the AAV rep and cap genes) in HEK293 cells. In this system we compared pAAV-RC-3Xneg in which the X ORF was fully incapacitated by having the three most 5′ ATGs knocked out (pAAV-RC-Xneg) to fully wild type pAAV-RC and found that both rAAV DNA replication and virion production were mildly inhibited by about half (statistically significant).

The level of replication boost provided by X appears to be most strong in differentiating keratinocytes and in HEK293+X versus normal HEK cells, yet in all cases the increase in DNA replication induced by X was statistically significant. Why there are differences in the strength of augmentation of the various forms of AAV2 replication we assayed for is presently unclear. As for the production of rAAV for use in gene therapy, all of the standard production schemes include the lip-cap gene and thus they also contain X (ending at nt 4393) which is fully located within lip-cap (ending at nt 4407). Transcripts originating from the p81 promoter, just up-stream from X, were confirmed by both S1 nuclease protection and primer extension (9).

### Materials and Methods

### Virus and Cells.

Cloned AAV2, pSM620, titered AAV2, and adenovirus type 5 viral stocks were originally obtained from Dr. Ken Berns. pAAV-RC-3Xneg was generated from pAAV-RC (Stratagene) by GenScript with mutations dictated as in FIG. 5B. pSM620-3Xneg was generated by replacing the BsiW I-SnaB I fragment (AAV2 sequence nt 3254-4497) of pSM620 with that from pAAV-RC-3Xneg. Dl63-78 (dl, deletion) was generated by ligating the appropriate Bgl II-Eco RV fragments from ins63 (ins, insertion of Bgl II linker) and ins78 (1). Dl78-91 was generated by ligating the appropriate Bgl II-Eco RV fragments from ins78 and ins91 (1). AAV/eGFP was generated by ligating the eGFP coding sequence into the Xho I site just behind the CMV promoter in dl3-97/CMV. Primary human foreskin keratinocytes were obtained from Clonetics. J2 (Meyers, 1996; Meyers et al., 1993) and HEK293, hereafter called 293 cells (Hermonat et al., 1997) cells have been described previously. Primary human foreskin keratinocytes (PHFK)(Clonetics) were maintained in keratinocyte SFM medium from GibcoBRL±Life Technologies (Cat. No. 10724-011). Epithelial organotypic rafts were maintained in E medium, which has been described previously (Meyers, 1996; Meyers et al., 1993). 293 cells were maintained in Dulbecco's modification of Eagle's medium with 7% fetal bovine serum and antibiotics.

### Transfection and Generation of Epithelial Organotypic Rafts.

Primary human foreskin keratinocytes (PHFK) were transfected with AAV/X/Neo or AAV/Neo plasmids (5 μg each, or as indicated) into 3×10⁵ PHFK using Fugene6 per manufacturer's instructions (day 0). The next day the cultures were infected with a multiplicity of infection (moi) of 100 AAV2 virus (day 1). The following day the cells were trypsinized and epithelial raft tissues were generated as described previously (11-13), with the exception that no protein kinase C inducers, such as TPA, were added to the culture medium. Briefly, 3×10⁵ of the transfected/infected PHFK were plated onto collagen disks containing J2 fibroblast cells submerged in E medium and the cells were allowed to adhere for 2 h and then the raft lifted to the air±liquid interface (day 2). The raised raft cultures were allowed to stratify and differentiate as previously described (11-13) and the experiment is depicted in FIG. 6B.

### Analysis of AAV DNA Replication in Epithelial Organotypic Rafts by Southern Blot

Total DNA was isolated from the raft tissues. The raft tissue was minced and placed in 500 ml of lysis buffer [5 mM Tris/HCl (pH 7.4), 5 mM EDTA, 0.25 mg/ml proteinase K]. After tissue was digested, the solution was phenol extracted and ethanol precipitated to purify total cellular DNA. For the measurement of AAV progeny formation by second plate amplification assay, after 36 h Hirt DNA was isolated from these second plate amplifications as previously described (11-13).

Analysis of AAV2 Rep and Cellular TF_{II}B mRNA Expression in Epithelial Organotypic Rafts by RT-PCR

Total RNA was isolated from rafts on day 5 using Trizol reagent (Invitrogen, Carlsbad, Calif.), according to the manufacturer protocol and treated with 5 U/Ag of RNase-free DNase I at 37-C for 2 h. Messenger polyA RNA then was isolated using the Oligotex mRNA Mini Kit (QIAGEN Inc. Valencia, Calif.) according to the supplier's instruction. The first-strand cDNA synthesis was performed at 37-C for 1 h in a final volume of 25 Al reaction buffer (1 Ag mRNA; 50 mM Tris-HCl, pH8.3; 75 mM KCl; 3 mM MgCl2; 10 mM DTT; 0.5 Ag oligo(dT)15; 0.5 mM each of the four dNTPs; 30 U of RNasin and 200 U of M-MLV Reverse Transcriptase RNase H Minus (Promega Co., Madison, Wis.)). PCR amplification (32 cycles) of the cDNA was performed in a 100-Al reaction volume which contained 2.5 U Taq DNA polymerase; 10 mM Tris-HCl, pH8.3; 50 mM KCl; 2 mM MgCl2; 0.2 mM each of the four dNTPs; 1 AM of each upstream and downstream primer specific for the cDNA template and 10 Al cDNA templates. The primer set used for AAV rep was 5V-TGAAGCGGGAGGTTTGAACG-3V and 5V-TCCATATTAGTCCACGCC-3V, which targeted amplification of the AAV sequences from nt 291 to 821. The TF_{II}B (housekeeping gene) was also analyzed in each RT-PCR mix. The products were size separated by agarose gel electrophoresis, stained with ethidium bromide and photographed.

### Analysis of AAV2 DNA Replication in 293 Cells.

293 (6 cm plates) cells at 70% confluence were transfected with 3 μg of the indicated plasmid. When the 293 cells were infected with Ad5 (moi 10) for helper function the cells were harvested at 2 days post-transfection. When the 293 cells were transfected with pHelper (Ad5 helper genes) and pAAV-RC (AAV rep and cap) plasmids, the cells were harvested on day 5. Cells were lysed with 1.5 ml of 1% SDS, 7.2 pH Tris-HCL, 5 mM EDTA, and Pronase K and incubated overnight. The total cellular DNA was then drawn though a 20 gauge needle ten times (to make less viscous), phenol extracted, ethanol precipitated twice, and 10 μgs of DNA were agarose gel electrophoresed, Southern blotted and probed with the indicated ³²P-labeled DNA probe. When, 2^{nd} plate virus production analysis was done, cells/medium were freeze-thawed three times, heated to 56° C. for 30 minutes, and 300 μls (or as indicated, from a total of 5 ml) was then used to infect a second plate of 293 (6 cm plates) cells at 70% confluence which were infected with Ad5 (moi 10). At two days post-infection total cellular DNA was isolated and analyzed by Southern blot as just described. After autoradiography densitometric analysis was carried out using the Alpha Imager 2000 with resident software (Alpha Innotech Corporation, San Leandro, Calif.).

### Virion DNA Analysis.

Six cm plates of transfected 293 cells were freeze-thawed three times, cellular debris pelleted by centrifugation at 7,000 rpm for 25 minutes, and the supernatant pushed through a 0.22 μm filter. Three hundred μl of virus stock was treated with 20 units DNase I for 30 minutes at 37° C. After heating the sample for 10 minutes at 100° C., the sample was digested with proteinase K (0.2 μg/ml) for 4 hrs, then phenol extracted and ethanol precipitated (with addition of 10 μg tRNA). The resulting DNA was then agarose gel electrophoresed, Southern blotted and probed with ³²P-eGFP DNA when analyzing for rAAV production or with ³²P-pSM620 DNA, when analyzing for wt AAV production.

### Infectivity Assay.

AAV/eGFP virus stock was equalized according to the relative titer determined by the densitometric analysis of DNase I-resistant virion DNA and 100-400 μls (equalized for amount of virus) of AAV/eGFP virus stock were used to infect 70% confluent plates of 293 cells. AAV/eGFP transduction was measured by eGFP fluorescence at 48 hours post-infection.

### Western Blot Analysis of X Protein.

Anti-38 rabbit polyclonal antibody was generated by GenScript against the peptide FRGPSGQRFHTRTDC, representing X sequences from aa 38-51. Total proteins were extracted from the 293 cells in the CelLytic™ M mammalian Cell Lysis/Extraction reagent (SIGMA). Protein concentration was determined using the protein assay dye reagent (Bio-RAD) and were normalized for equal loading. After separating on 10% SDS-PAGE gels, protein was transferred to Immun-Blot PVDF membranes. The membranes were then blocked for 1 hour at room temperature with 5% nonfat milk in 1×TBST buffer (10 mM Tris-Cl (pH 7.5), 150 mM NaCl, 0.1% Tween 20). Followed a brief rinse, membranes were incubated with polyclonal anti-38-X(horseradish-peroxidase (HRP)-conjugated antibody (1:500 dilution, Sigma-Aldrich) at 4° C. overnight. Washes in 1×TBST buffer were performed between incubations for three times. Blots were developed with Pierce® ECL system (Thermo-Fisher Scientific). Probe detection of β-actin was carried out as control.

### Example 3

### AAV “X” is an Oncogene

In cell transfection experiments we noticed that the transfection of “X”-expressing plasmid caused the medium of cells to become yellow (acid pH, higher metabolic activity) before that of control plates. Thus we considered that “X” may be a possible oncogene or pro-growth gene. We found AAV2 “X” has homology to HTLV2 Tax, a known oncogene (49-51), and to cellular INO80, a protein involved in chromatin remodeling (44-48) and known to bind p53 (45). So we tested “X” for oncogenic transformation abilities in contact inhibited swiss albino 3T3 cells.

### Results

### Introduction of X Causes Focus Formation in Contact-Inhibited Swiss Albino 3T3 Cells (SA3T3)

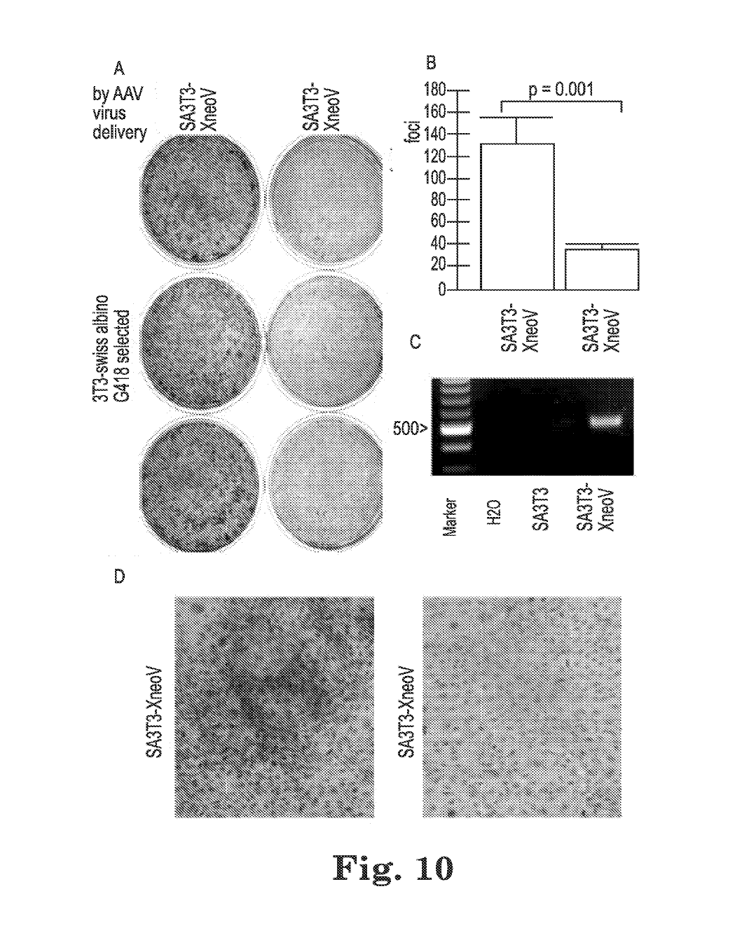

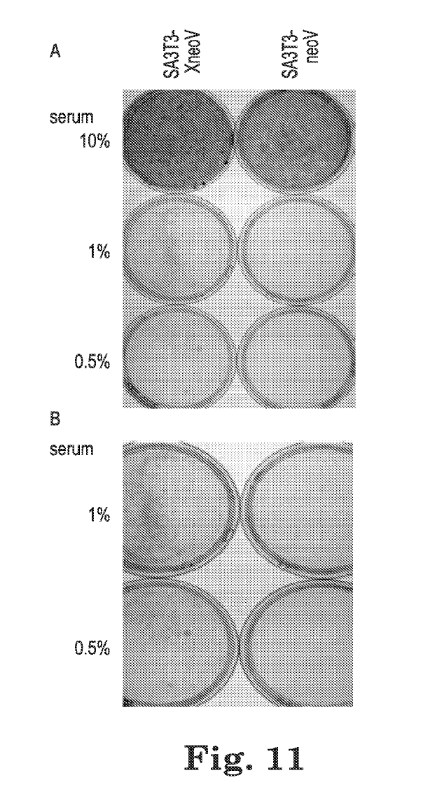

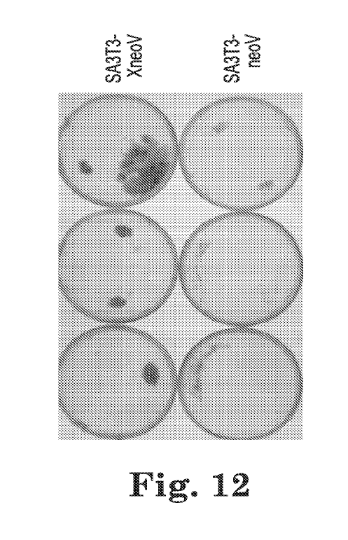

While the function of X is unverified, one possible function might be that of a progrowth gene, possibly even an oncogene, as all other small DNA viruses encode at least one such gene. To test this hypothesis initially we infected several contact inhibited rodent fibroblast cell lines and found little evidence of loss of contact inhibition/oncogenic transformation. It was next considered that AAV2 X may be a weak oncogene, and perhaps X needed a more sensitive assay for observing its potential oncogenic phenotype. Therefore, we then infected SA3T3 cells with AAV/X/Neo and AAV/Neo virus and selected for the genetically altered/transduced cells by G418-resistance, to give SA3T3-XneoV and SA3T3-neoV respectively. The resistant colonies were then replated, allowed to reach confluence, and then fixed and methylene blue stained at 15 days post-confluence. It can be seen in FIG. 10 panels A and B that the SA3T3-XneoV gave rise to transformed foci, whereas the SA3T3-neoV cells gave statistically fewer foci. In FIG. 10 panel C it is further shown that representative SA3T3-XneoV cells contained the AAV2 X gene as determined by PCR amplification. Finally, in FIG. 10 panels D and E representative photomicrographs of SA3T3-XneoV, SA3T3-neoV cells are shown. Note that the density of the SA3T3-XneoV cells were much higher, with cell piling (foci), than the SA3T3-neoV control cells.

### Introduction of X Causes Lower Serum Requirement

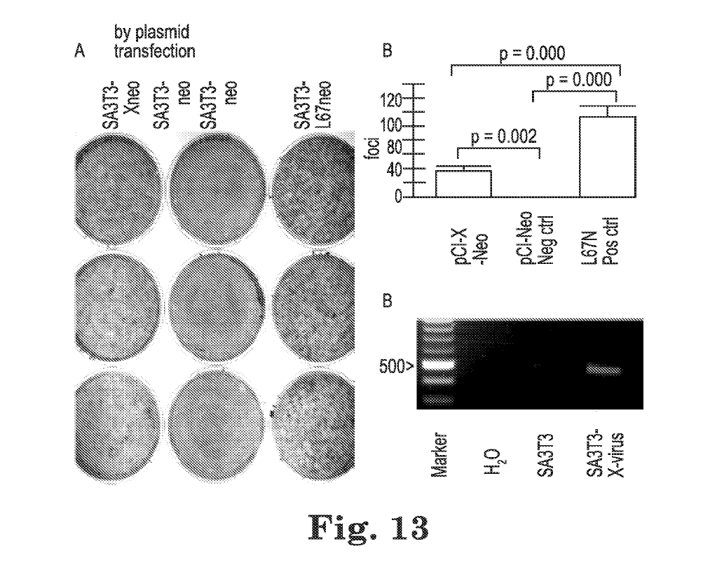

These same cells were than analyzed for fetal bovine serum (FBS)-independent growth. Five×10⁴ X-positive cells and X-negative SA3T3 cells were plated and fed with medium with decreased amounts, ranging from 10% to 0.5% FBS and fixed and stained at 14 days post-plating. As shown in FIG. 11 panel A the SA3T3-XneoV cells were able to grow faster in low serum, both in 1% and 0.5% FBS, than the X-negative SA3T3-neoV cells. An enlargement of these cells is shown in FIG. 11 panel B to more easily see the differences in growth. Thus the presence of the X gene was associated with lower serum dependence. Like the foci experiment (FIG. 2), these data are also consistent with a progrowth/oncogenic phenotype for AAV2 X.

### Introduction of X Causes Higher Invasion

The cells were then analyzed for their ability for growth in soft agar. Five×10³ of the X-positive and X-negative SA3T3 cells were mixed and plated into soft noble agar with DMEM and fed with an overlay of 10% DMEM/10% FBS. We were surprised that after two weeks we could not observe any colony formation in the agar of any plate, either SA3T3-XneoV or SA3T3-neoV by microscopic evaluation. Yet when we formalin fixed the plates it was noticed that quite a few cells had become attached to the plastic plate outside of the agar, after accidently losing one of the agar plugs from the dish (it fell out when the medium was being decanted). Thus, we removed all of the agar plugs and methylene blue stained the remaining attached cells. The results are shown in FIG. 12. It was apparent that some cells were able to migrate out of the agar, attach to the plate's surface, and continued to grow. As can be seen, the presence of X was associated with much higher levels of out-of-the-agar invasion. This attribute is yet another phenotype associated with oncogenicity, again attributed to the X gene.

AAV2 X Gene Causes Focus Formation, but Less than HPV E6-E7

Next calcium phosphate transfection was carried out to analyze the pro-growth/oncogenic properties of X. The plasmid pCI-Neo, was a negative control compared and compared to a positive control, pL67N. The pL67N plasmid contains the human papillomavirus type 16 (HPV-16) long control region (LCR) and the down-stream E6 and E7 oncogenes, with the Neomycin resistance gene (Neo) ligated just downstream of the E7 gene (34).

pCI-X-Neo was the X-positive experimental plasmid. After transfection into SA3T3 cells the three transfected cells groups were G418 selected, replated, and allowed to reach confluenece, and at two weeks post-confluence the cells were fixed and stained. The SA3T3 cells containing only Neo, X, or E6-E7 were referred to as SA3T3-neo, SA3T3-Xneo, and SA3T3-L67neo, respectively.

The resulting cells are pictured in FIG. 13 panel A and a visually quantification for foci (FIG. 13 panel B) indicates that the SA3T3/L67N cells resulted in the highest number of foci, followed by SA3T3/pCI-X-Neo cells, and the SA3T3-pCI-Neo cells gave no foci. Moreover, the SA3T3/pCI-X-Neo cells were shown to contain X DNA by PCR amplification (FIG. 13 panel C).

AAV2 X Gene is Packaged into Virions without Covalently-Attached ITRs

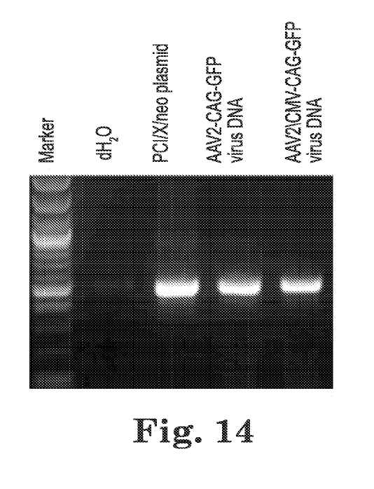

Having shown that AAV2 X was pro-growth/oncogenic properties, causing foci on SA3T3 cells by both viral and calcium phosphate transfection, we were also aware that during the generation of recombinant rAAV2 by plasmid transfection, that plasmid DNA was packaged into AAV virus particles, and as high as 6% of those virions (35-38), Thus we needed to identify if the AAV2 X DNA from the helper plasmids such as pAAV-RC, was specifically packaged to give X-positive rAAV2. Therefore, we purchased cesium chloride gradient purified AAV2-CAG-GFP virus, generated our own AAV2/CMV-eGFP virus, and isolated DNase I-resistant virion DNA from both these virus stocks. These virion DNA samples were analyzed for the presence of AAV2 X DNA. PCR primers designed to to amplify the full length of the AAV2 X reading frame were used and the results compared to control dH₂O (negative control) and to pCI-X-Neo plasmid DNA (positive control). As can be seen in FIG. 14 both rAAV2 virus stocks were shown to contain X DNA. Thus, even though X sequences were only located in the helper plasmid, without covalently attached inverted terminal repeat DNA, full length X DNA was still packaged into AAV2 virions.

### Discussion

This study demonstrates that AAV2 X has progrowth/oncogenic actuvuties on swiss albino 3T3 cells. Although most virologists studying parvoviruses/dependoviruses will consider this report surprising, it should be obvious that AAV would need such a pro-growth/oncogene as all small DNA viruses other than parvovirues, in fact, encode such genes. Within hepadnaviruses, such as hepatitis B virus, studies suggest that it is the HBx gene which is the likely oncogene (39-41). The exact mechanism of action of HBx has not yet been determined. Within the polyomaviruses, simian virus type 40 (SV40), its large tumor antigen (T antigen) causes malignant transformation of cells through the binding of both retinoblastoma (Rb105) and p53 ant-oncoproteins. Additionally, two human polyomaviruses, BK and JC virus, have been shown to be oncogenic in both rodents and primates (43). A subset of human papillomaviruses (HPV, high risk HPV types, are found at high frequency in a variety of human cancers. Among the high risk HPV types, HPV16 and HPV18 are the principal causes of cervical cancer (44). It is characteristic of these cancers that the HPV DNA is found to be chromosomally integrated into the cancer cell's genome. There are two main viral oncoproteins which are involved in cervical carcinogenesis. These are E6 and E7, which bind and inactivate two very important cellular tumor suppressors, p53 and Rb105, respectively (45). There is also the E5 oncoprotein which is less understood (46). While not linked to human cancers, it is clear that many of the adenovirus (Ad) serotypes, including types 2, 5,12, 18, and 31, are able to oncogenically transform contact-inhibited murine cells in culture and induce tumors in hamsters and rats (47-49). Ad has two major viral oncogenes, E1A and E1B, have been identified oncoproteins as responsible for adenovirus tumorigenicity, which bind and inactivate Rb105 and p53, respectively (47-49). However, E4orf6 may also have oncogenic potential (45). Thus, actually, it should not be surprising at all that AAV2 also encode something like an oncoprotein.

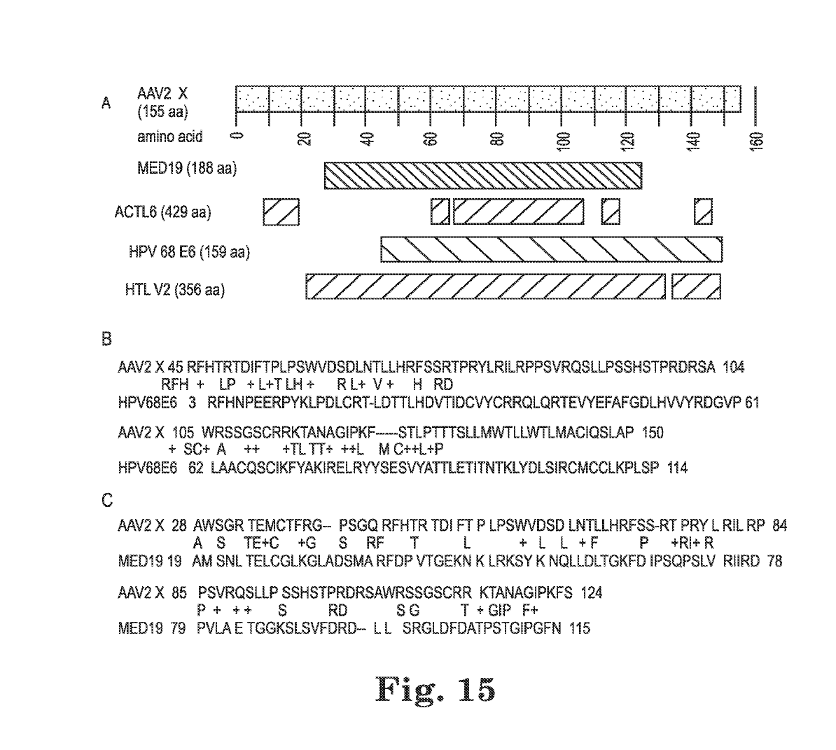

Upon carrying out Protein Blast (National Center for Biotechnology Information) search for homology with other proteins, it was found that AAV2 X had homology to many interesting and important proteins. These included polymerases and accessory proteins, helicases, topoisomerases, and many types of DNA binding proteins. Actin-like protein 6 (Baf53/ACTL6/INO80) was at the top of the list of homologous cellular proteins to AAV2 X (50,51). However, human ACTL6 is considerably larger at 429 amino acid (aa) residues than AAV2 X (155 aa). Thus, ACTL6 may not be the most accurate model for suggesting what X does. Searching further, in particular smaller proteins, X was found to have homology fungal RNA polymerase II transcription subunit 19 (MED19, Rox3), also a relatively small protein (181 aa)(28). Homology with MED19 was found across 62% of X (see FIG. 15). It is important to note that the human homologue of fungal MED19 is lung cancer metastasis-related protein 1 (LCMR1) and is known to be an oncogene in human lung and other cancers (53). Thus, as a starting point, noting both homology and size, MED19/LCMR1 is perhaps one model for X activity which should be considered.

Searching further still, we observed proteins homologous to X among viral proteins, such as, X has homology with human T-cell lymphotropic virus 2 (HTLV2) Tax (FIG. 15 panel A) (24,25). The homology of Tax with X was seen across a wide expanse of the X protein. HTLV1 Tax was less homologous. FIG. 15 panel A shows the regions of homology between HTLV2 Tax and AAV2 X. However, Tax is over twice the size of X, at 331-356 aa). Another homologous viral protein was HPV E6, particularly that of type 68, as seen in FIG. 15 panel A. HPV68 is considered a “high risk” type for cervical cancer and, while HPV68 E6 has not been specifically studied, the E6 protein usually has the ability to bind p53 (32). Additionally, and important, E6, at 158 aa in length, is very similar in size to AAV2 X. The homologies shown by NCBI Protein blast between X and the smaller oncoproteins MED19 and E6 are shown in FIG. 15 panels B and C, respectively.

One common comment usually made when showing these data to others has been “why hasn't anyone seen evidence of this before”? One reason may be that AAV2 also encodes an anti-oncoprotein Rep78 (also a replication protein), and its presence likely significantly masks the effects of X (28, 55, 56). The hypothesis of an interplay between Rep78 and X has some merit. For example the p81 promoter, from which X is expressed, may be a Rep-dependent promoter as are all the other AAV promoters (57) and we have preliminary data showing Rep78 binding to p81 DNA (data not shown). In any case, we hypothesize that X is an AAV2-encoded pro-growth protein and is involved in the life cycle of AAV similar to how the pro-growth proteins of the other small DNA viruses are involved in the life cycle of those viruses. In retrospect, the possibility of a growth-promoting gene is not so unexpected as all other small DNA viruses encode such genes.

These data further strongly suggest that X is a real gene, encoding a real protein, and has an likely has an important role in stimulating cell division for enhancing the completion of the AAV2 life cycle, but also that X may be a safety hazard.

### Materials an Methods

### Virus and Cells

AAV/X/Neo was generated by cloning the AAV2 X ORF behind the p5 in AAV vector AAV/Neo. AAV/X/Neo, AAV/Neo, and AAV/CMV-eGFP virus were generated by co-transfection of the vector plasmid with pAAV-RC and pHelper plasmids into 293 cells and tittered by standard dot blot methodology (37-39). AAV2-CAG-GFP virus was purchased from Vector Biolabs (cat #7072). Swiss albino 3T3 (SA3T3) cells (American Type Culture Collection, CCL-92) were maintained in Dulbecco's Modified Eagle's Medium supplemented with 10% fetal bovine serum (FBS) and antibiotics. One×106 SA3T3 cells were infected with either AAV/X/Neo or AAV/Neo virus (moi 500), then G418 selected (400 μg/ml the first week, then half that afterwards) to give bulk G418 resistant cell lines give SA3T3-XneoV and SA3T3-neoV. Similarly SA3T3 cells were calcium phosphate transfected (CalPhos Mammalian Transfection Kit, Clontech, Cat #631312) with AAV/X/Neo, AAV/Neo, or L67N plasmids, G418 selected for two weeks, to give SA3T3-Xneo, SA3T3-neo, SA3T3-L67neo cells, respectively.

### Western Blot Analysis for X Protein

Anti-aa38 rabbit polyclonal antibody was generated by GenScript against the peptide amino-FRGPSGQRFHTRTDC-carboxy, representing X sequences from aa38-51. 293 cells were transfected with pSM620 plus AAV/Neo or AAV/X/Neo plasmids and then infected with Ad5 at an moi of 5. After 48 hours total cellular proteins were extracted from the cells using the CellLytic™ Mammalian Cell Lysis/Extraction reagent (SIGMA). Protein concentration was determined using the dye reagent (Bio-RAD) and were normalized for equal loading. After polyacrylamide electrophoresis (10% SDS PAGE gel), protein was transferred to Immun-Blot PVDF membranes. The membranes were blocked for 1 hour at room temperature with 5% nonfat milk in 1×TBST buffer (10 mM Tris-HCl (pH 7.5), 150 mM NaCl, 0.1% Tween 20. Following a brief rinse, membranes were incubated with polyclonal anti-38-horseradish-peroxidase (HRP)-conjugated antibody (1:500 dilution, GenScript) at 4° C. overnight. The next day, the membranes were washes 3× in TBST buffer, each for 10 minutes. Blots were developed with Pierce® ECL system (Thermo-Fisher Scientific).

### Analysis for Focus Formation

The SA3T3-XneoV, SA3T3-neoV, SA3T3-Xneo, SA3T3-neo, SA3T3-L67neo mixed bulk colony cell lines, after G418 selection, were split into 6 cm plates at 5×10⁵ cells per plate, allowed to reach confluence, fed every three days afterwards, and formalin-fixed/methylene blue stained at 14-16 days post-reaching confluence. Foci were counted by visual inspection.

### Analysis of Cell Serum Requirements

The SA3T3-XneoV and SA3T3-neoV cells were compared for their ability to grow in reduced serum. A total of 5×10⁵ cells of each type were plated into 35 mm plates in DMEM plus 10%, 1%, or 0.5% FBS. After 12 days the cultures were formalin fixed and methylene blue stained.

### Analysis of Cell Invasion.

The SA3T3-XneoV, SA3T3-neoV cells were plated in double-layered agar, in triplicate, to determine the frequency of soft agar colony formation in the cell population. Briefly, 2 ml of 42° C., 0.4% soft agarose/complete culture medium were added to 5×10⁴ of the cells, gently pipetted up and down to mix and put onto 35-mm dishes with a 0.8% soft agarose underlay. The agarose was allowed to harden for 10 min and then incubated at 37° C., 5% CO₂ for 14 days. The number of colonies larger than 0.5 mm diameter was then counted using an inverted microscope (none were found). Additionally, the plates were fixed with DMEM/4% formaldehyde, the agar plug removed, and the remaining attached cells were stained with methylene blue.

### Virion DNA Analysis for X.

Three hundred ul of virus stock (AAV2-CAG-GFP and AAV/CMX-eGFP) were treated with 20 units DNase I for 30 minutes at 37° C. After heating the sample for 10 minutes at 100° C., the sample was digested with proteinase K (0.2 ug/ml) for 4 hrs, then phenol extracted and ethanol precipitated (with addition of 10 pg tRNA). PCR amplification (32 cycles) of the virion DNA was performed in a 100-pl reaction volume which contained 2.5 U Taq DNA polymerase; 10 mM Tris-HCl, pH8.3; 50 mM KCl; 2 mM MgCl2; 0.2 mM each of the four dNTPs; 1 uM of each upstream and downstream primer specific for the DNA template The primer set used for AAV X from nt3929 to 4396 using primers X-up: 5′-ATCTCGAGAGCAGTATGGTTCTGTATCTACC-3′ and X-down: 5′-AGTCGACATTACGAGTCAGGTATCTGGTG-3′ which amplifies the full length X ORF sequences. The products were size separated by agarose gel electrophoresis, stained with ethidium bromide and photographed.

### Example 4

### AAV2 X Helps Replication of a Different AAV Strain

### Introduction

Now there are over 100 adeno-associated virus (AAV) types isolated. While AAV type 2 (AAV2) was the first AAV type used for gene transfer (Hermonat, 1984, 2014; Hermonat and Muzyczka, 1984, Hermonat et al, 1984), but over time more and more AAV types, each with its own somewhat different cellular tropisms, are coming into use. In general these other AAV types have the same genomic structure as AAV2 (Gao et al, 2005; Srivastava et al., 1983). Analysis of the first cloned adeno-associated virus AAV type 2 (AAV2) genome showed that there were two main open reading frames (ORFs) and mutation within the identified ORFs indicated three trans phenotypes were present (Hermonat, et al., 1984). Mutations in the left half of the genome were defective in DNA replication and transcription and given the rep phenotype. This region encodes replication/transcription factor proteins Rep78, Rep68, Rep52, and Rep40. Mutations within the right half of the genome were defective in virion production and this, but the region had two phenotypes. One was given the name lip for the production of viral particles of low infectivity (missing VP1), while the cap phenotype didn't produce any viral particles at all (encoding the major structural protein, VP3) (Hermonat et al., 1984). Additionally, recently, a new fourth trans phenotype, involved in virion maturation, has been identified by Jurgen Kleinschmidt and called the AAP gene (Sonntag et al, 2010).

In this patent application, we disclose a fifth phenotype, a new gene we called X, within the AAV2 genome. The X gene is located at the carboxy-end of the cap gene but in a different translational frame. We have shown that X is needed for maximal wt AAV2 and rAAV2 DNA replication and virion production by several methods. The X gene also has a dedicated promoter located just upstream, called p81 (at map unit 81). However, the question arises is AAV2 X activity only specific for helping/augmenting AAV2, or is it capable of helping other AAV types? Most other AAV clades also have members with an open reading frame (ORF) in the same position as AAV2 X, but these potential genes are usually smaller than AAV2 X (to be reviewed, submitted elsewhere). Here we observed that AAV2 X is able to augment or boost an rAAV production system based exclusively on the AAV6 rep and cap, trans sequences and we find that X is capable of increasing rAAV2 DNA replication and virion production when driven by the AAV6 rep and cap genes. Additionally, we hypothesize that AAV2 X may be derived from a 5′/amino region of the AAV Rep78/NS1 gene.

### Results.

AAV6 Genome Contains an X Gene but which is Divided into Two Abutting ORFs.

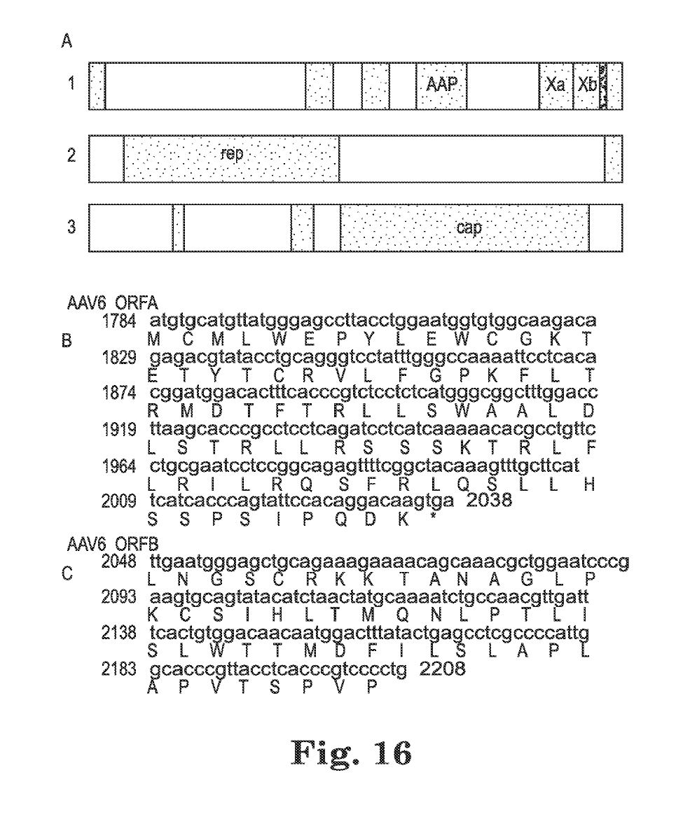

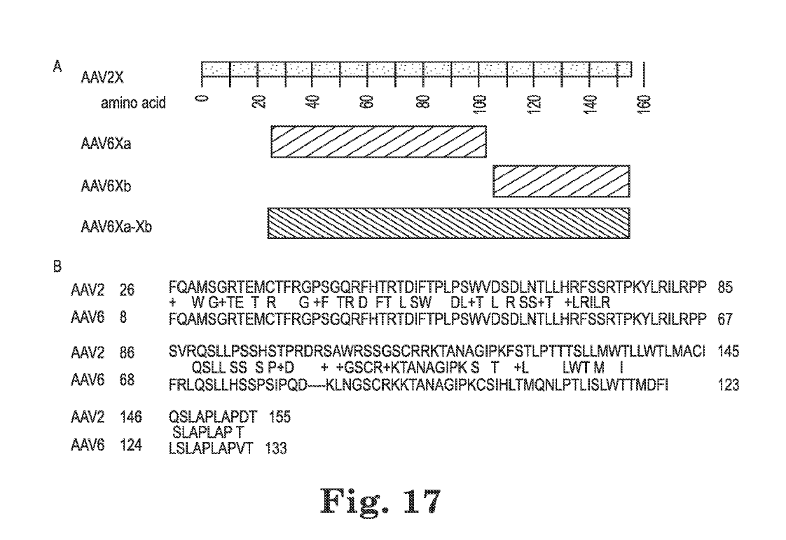

If one observes the open reading frames of the prototype AAV6 genome (Genbank AF028704) it is observed that there are two ORFs, which we refer to as Xa and Xb, which take up the position analogous to where the AAV2 X gene is. There is a small gap between the stop codon of Xa and the initiation codon of Xb. However, analyzing two other AAV6 sequences, specifically Genbank EU368909 and EU36910, there is an even smaller gap between Xa and Xb of only 13 nucleotides, and the Xb ORF encodes a further 22 amino acids (aa) at its amino terminus. FIG. 16 panel A shows the gene/ORForganization of AAV6 using largely the AF028704 prototype sequences, but with the X region of EU368909 replacing the analogous sequences of the prototype. FIG. 15 panels B and C show the DNA and amino acid sequences of Xa and Xb. FIG. 17 is a homology analysis by standard NCBI Protein BLAST of the amino acid sequence of AAV2 X versus those of the fused Xa and Xb aa of EU368909. As can be seen there is significant homology between the two X sequences across their length. This extensive homology suggests that AAV6 Xa-b is a homologue of AAV2 X and it has either evolved or mutated at some point in time. Presently, it is unknown if AAV6 Xa and Xb represent two potentially functional proteins or are fully inactive and “broken”. In any case AAV6 appears to have or have had a very AAV2 “X”-like protein.

AAV2 X Helps rAAV2/6-eGFP DNA Replication and Virion Production.

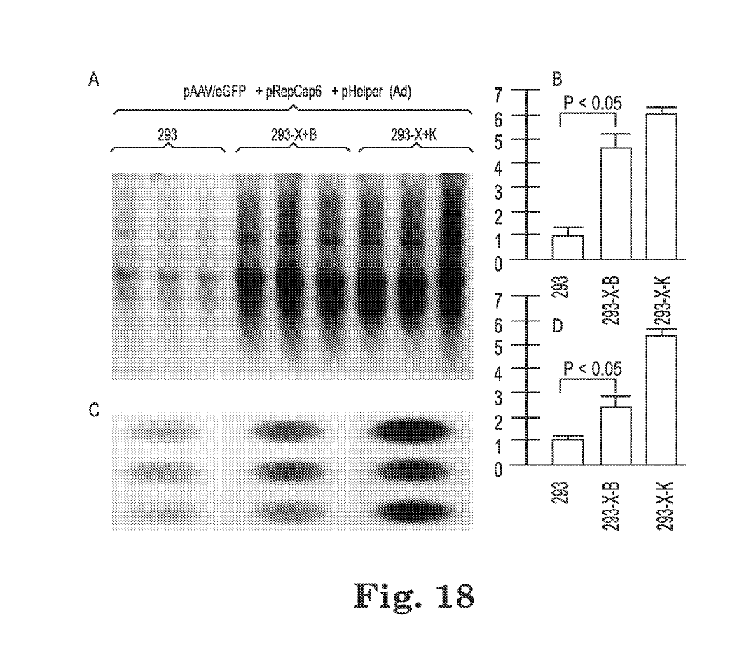

As we know that AAV2 X increases rAAV2 yield, and AAV6 X may be non-functional, we investigated whether AAV2 X might complement AAV6 rep/cap driven rAAV production. Previously we generated HEK293 cell lines containing chromosomal AAV2 X (293-X-B and 293-X-K) and we compared them to parental HEK293 cells for supporting rAAV2 DNA replication and virus production. Shown in FIG. 18 panel A is a Southern blot of rAAV2/eGFP DNA replication (probed with ³²P-eGFP DNA) by transfecting the vector plasmid with AAV6repcap and pHelper (Ad5 helper genes) plasmids. FIG. 18 panel B shows a dot blot of DNaseI-resistant virion DNA which shows higher rAAV production in X-positive 293-X-B and 293-X-K than in unaltered 293 cells. Moreover the higher virion production mirrors the higher vector DNA replication levels. As can be seen the presence of the AAV2 X gene in the B and K cell lines was able to boost rAAV production in the presence of the AAV6 rep and cap proteins as it did for rAAV with AAV2 rep and cap driving vector production.

AAV2 X Helps rAAV2/6-Foxp3 DNA Replication and Virion Production.

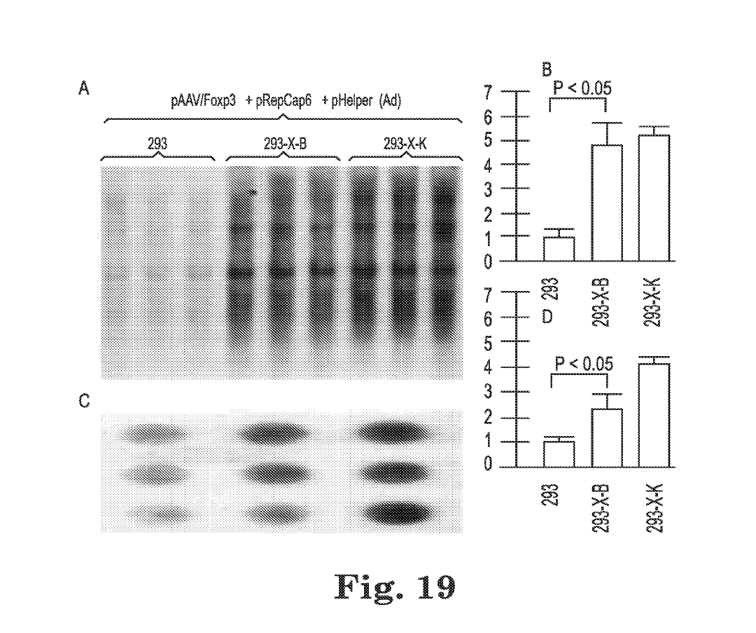

Similar experiments were done with the vector rAAV2/Foxp3 in place of AAV2/eGFP. FIG. 19 panel A shows a Southern blot of rAAV2/Foxp3 DNA replication (probed with ³²P-Foxp3 DNA) by transfecting the vector plasmid with AAV6repcap and pHelper (Ad5 helper genes) plasmids. FIG. 19 panel B then shows a dot blot of DNaseI-resistant virion DNA which shows higher rAAV production in X-positive 293-X-B and 293-X-K than in unaltered 293 cells. Again, as with the AAV2/eGFP vector, the presence of AAV2 X in the 293 cells, in conjunction with AAV6 rep and cap proteins, boosted vector rAAV2/Foxp3 DNA replication and virion production as it did for rAAV driven by AAV2 rep and cap.

### AAV2 X has Homology to the Rep78 Proteins of Various AAVs.

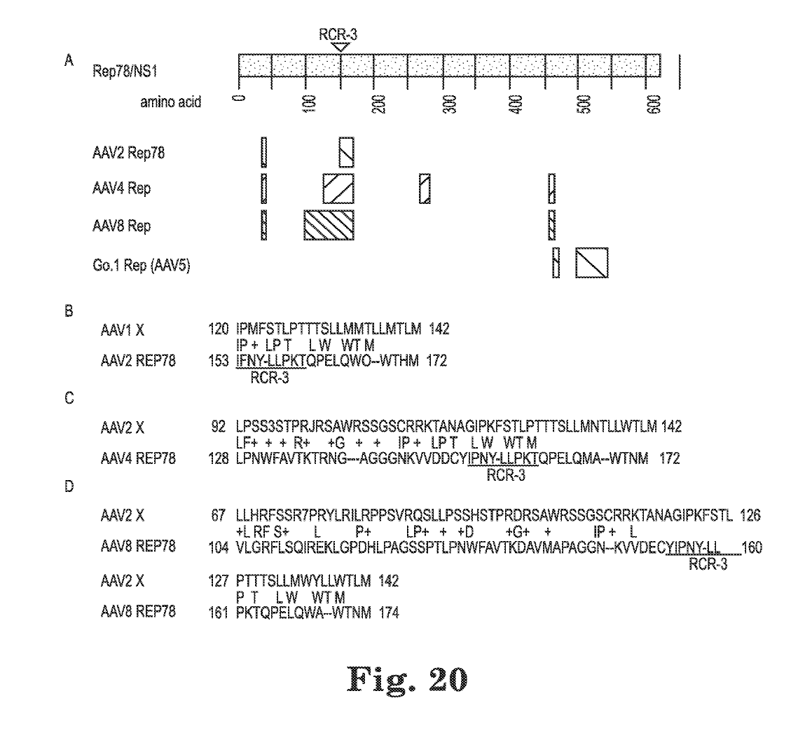

It was noticed during various NCBI Protein Blast searches that X showed homology with Rep78/NS1 of AAV2, but also other AAVs as well. Therefore in FIG. 20 panels A, B, C, and D we show the results of homology analyses with AAV2, AAV4, AAV8 and Go.1. The largest region of homology is seen between AAV8 Rep78 and AAV2 X It can be seen that most homology with X lies in a region from about aa100-200 of the Rep78 protein. However the results were quite different for Go.1, where homology with X was seen at the extreme carboxy-terminus. Clearly this finding is consistent with AAV2 X being involved in AAV DNA replication.

### Discussion.

In Example 2 it is demonstrated that AAV2 X boosts rAAV production driven by the AAV2 rep and cap genes/proteins (Hermonat et al, 1984; Hermonat and Muzyczka, 1984). In the present example, it is shown that AAV2 X also boosts rAAV production driven by AAV6 rep and cap proteins—the rep and cap proteins of a different strain of AAV. It is not surprising that AAV2 X helps AAV6, as the AAV2 and AAV6 Rep78 proteins are 89% homologous. Thus whatever role AAV2 X serves in relation to AAV2 Rep78 would likely still be active with substitution of AAV6 Rep78. In any case, these data suggest that AAV2 X is a prototype gene of a type which is widely present within dendoviridae. We are preparing a review of X homologues among dendoviruses, however there are still related issues that can be discussed at this time. First among these is what AAV X might do? Protein homologies provide the first evidence for how a protein may function. To this end various protein homologies were identified by Nation Center for Biotechnology Information (NCBI) Protein Blast analysis. Upon investigative search it was apparent that X has homology to two different gene types within various AAV2 isolates and other AAV types.

One homologous sequence to X within various AAV genomes is the cap (capsid) encoded proteins, VP1-3. However, this seems to be likely due to the knowledge that not all AAV type sequences are fully vetted (the prototype AAV2 sequences has been updated many times). Thus, as X is fully contained within the cap gene a single base addition or subtraction would fuse or splice X coding sequences into the capsid protein or visa versa. Only continued vetting of AAV genomic sequences can solve this issue. The second common homologous sequence to AAV2 X within various AAV isolates is with the rep (replication) encoded protein, Rep78. This X-to-Rep78 homology is shown in FIG. 20. As can be seen, FIG. 20 panels B-D show homology with AAV serotypes 2, 4, and 8 Rep78s (NC_001401.2, NC_001829.1, NC_006261.1, respectively). These homologies are present within the amino third of Rep78. AAV8 Rep78 has the most extensive homology, over a 70 aa region, about half of AAV X These X homologous are shown graphically against the background of a generalized Rep78 protein in FIG. 20 panel A. Thus, drawing directly from these homologies, AAV2 X may be derived by some type of non-homologous recombinant exchange from the 5′ half of the rep region and the 3′ portion of the cap region of AAV. If this happened did happen most likely this exchange could be hypothesized to have taken place between the rep gene of AAV8 and the cap gene of AAV2. Moreover the same region of Rep78 from several AAV types is homologous to AAV2 X (FIG. 20). This region of homology contains a portion of AAV2 X identified as being a DNA binding region by AAAAA. The Rep78 helicase requires two Rep-Rep binding sites for hexameric-association on DNA, one in the amino-terminal region and another within the carboxy-two thirds. That X contains significant homology to the amino-hexamer-DNA binding domain of Rep78 suggests the possibility that X might bind to itself or to Rep78 in the presence of DNA (or even possibly without). Additionally, if X were able to associate with Rep52/Rep40 the resulting heterodimers might reconstitute additional biochemistries of the full length monomers of Rep78/Rep68 proteins. Many interesting possibilities exist, for example, might X interact with Rep78-ITR complexes and modulate their activities? Additionally, this region of X has homology with the rolling circle replication region 3 (RCR3) of Rep78 which is believed to be involved with cutting and ligating single stranded inverted terminal repeat DNA (Smith and Kotin, 2000). Thus there is the possibility that any of these mentioned activities of Rep78 might be either augmented or inhibited by AAV2 X.

It is not fully surprising that AAV2 X helps AAV6 as the AAV2 and AAV6 Rep78 proteins are 89% homologous (NC_001401.2; AF028704.1, respectively). Additionally AAV2 X has some level of homology with AAV large Rep proteins, in particular with the AAV8 Rep78 equivalent. FIG. 20 shows that homology which is identified by Nation Center for Biotechnology Information (NCBI) Protein Blast analysis. FIG. 20 panels A-C shows the homology between AAV2 X and Rep78. The information that AAV2 X helps AAV2 and 6 Rep78 replication activity and has homology in a specific region of many Rep78s suggests the possibility that X and Rep78 may be interacting partners, giving a new Rep78-X hetero-dimeric with new or accentuated activities. However, there is another homologous partner to X, that being the Rep78 (NS1) protein of Go.1/AAV5 (DQ335246.2). This segment of homology has a very different location within Rep78, being between AAV2 X and the carboxy-terminus of Go.1, a relative of AAV5. Finally, as X augments both AAV2 and AAV6 rep/cap driven rAAV production, it would seem likely that other AAV types, besides just AAV6 and 2, would also be helped by AAV2 X

### Materials and Methods

### Virus and Cells.

HEK293 cells were maintained in Dulbecco's modification of Eagle's medium with 7% fetal bovine serum and antibiotics. The HEK293 cell lines, 293-X-B and 293-X-K cell lines have been described previously. AAV/eGFP was generated by ligating the enhanced green fluorescent protein (eGFP) coding sequence into the Xho I site just behind the CMV promoter in dl3-97/CMV. AAV/Foxp3 was generated in a similar manner.

### Analysis of AAV2 DNA Replication in 293 Cells.

HEK293, 293-X-B, or 293-X-K cells (6 cm plates) at 70% confluence were transfected with 1 μg of the indicated vector plasmid (AAV/eGFP or AAV/Foxp3 plus 1 μg pRepCap6 plus 1 μg of pHelper(Ad) using TransIT according to manufacture's instructions. For DNA replication analysis cells were lysed with 1.5 ml of 1% SDS, 7.2 pH Tris-HCL, 5 mM EDTA, and Pronase K and incubated overnight. The total cellular DNA was then drawn though a 20 gauge needle ten times, phenol extracted, ethanol precipitated twice, and 10 μgs of DNA were agarose gel electrophoresed, Southern blotted and probed with the indicated ³²P-labeled DNA probe. After autoradiography densitometric analysis was carried out using the Alpha Imager 2000 with resident software (Alpha Innotech Corporation, San Leandro, Calif.).

### Virion DNA Analysis.

Six cm plates of transfected HEK 293, 293-X-B and 293-X-K cells, were treated as in the analysis of DNA replication. After three days cells were freeze-thawed three times, cellular debris pelleted by centrifugation at 7,000 rpm for 25 minutes, and the supernatant pushed through a 0.22 μm filter. Three hundred μl of virus stock was treated with 20 units DNase I for 30 minutes at 37° C. After heating the sample for 10 minutes at 100° C., the sample was digested with proteinase K (0.2 μg/ml) for 4 hrs, then phenol extracted and ethanol precipitated (with addition of 10 μg tRNA). The resulting DNA was then dotted blotted onto a nylon membrane and probed with either ³²P-eGFP or ³²P-Foxp3 DNA, as appropriate, when analyzing for rAAV production.

### Example 5

### Homology of AAV2 X with Possible X Genes in Other AAV Strains

The amino acid sequence of the product of the X gene of AAV2 was compared with the translated protein sequence of open reading frames in other AAV strains to identify possible X genes in other AAV strains. The results are shown in Table 2.

TABLE 2
Comparison of AAV2 X with those of other clades

| | repre- | | | |
|---|---|---|---|---|
| | senta- | | | |
| Clade | tive | Identities | Positives | Gaps |
| Clade | AAV1 | 37/77 (48%) | 46/77 (60%) | 0/77 (0%) |
| A | OrfA | | | |
| Clade | hu.29R | 155/155 (100%) | (100%) | 0/155 (0%) |
| B | | | | |
| Clade | hu.11 | 21/42 (50%) | 24/52 (57%) | 0/42 (0%) |
| C | | | | |
| Clade | cy.5R4 | 58/97 (60%) | 65/97 (67%) | 8/97 (8%) |
| D | OrfB | | | |
| Clade | cy.5R4* | 80/142 (56%) | 91/142 (64%) | 12/142 (8%) |
| D* | | | | |
| Clade | AAV8 | 28/66 (42%) | 38/66 (58%) | 1/66 (1.5%) |
| E | | | | |
| Clade | AAV9 | 22/50 (44%) | 29/50 (58%) | 0/50 (0%) |
| F | | | | |
| AAV3 | AAV3 | 22/40 (45%) | 26/44 (59%) | 2/44 (5%) |
| AAV5 | Go.1 | 4/9 (44%) | 5/9 (44%) | 0/9 (0%) |
| Clade | | | | |
| AAV10 | AAV10 | 25/38 (66%) | 26/38 (68%) | 0/38 (0%) |
| AAV12 | AAV12 | 17/34 (50%) | 18/34 (53%) | 0/34 (0%) |
| Rh.39 | Rh.39 | 48/77 (62%) | 55/77 (71%) | 0/77 (0%) |
| Hu.T88 | Hu.T88 | 141/155 (91%) | 145/155 (93%) | 0/155 (0%) |

*Combining ORFA and B

### REFERENCES

1) Hermonat, P. L., Labow, M. A., Wright, R., Berns, K. I., and Muzyczka, N. (1984) Genetics of adeno-associated virus: isolation and preliminary characterization of mutants in adeno-associated virus type 2. J. Virology 51(2):329-339.
2) Hermonat, P. L., and Muzyczka, N. (1984) Use of adeno-associated virus as a mammalian DNA cloning vector: transduction of neomycin resistance into mammalian tissue culture cells. Proc. Natl. Acad. Sci. U.S.A. 81:6466-6470.
3) Hermonat P L. Genetic analysis and utilization of adeno-associated virus as a mammalian cloning vector. Univ F L Dissertation.
4) You C X, Shi M, Liu Y, Cao M, Luo R C, Hermonat P L. (2012) AAV2/IL-12 gene delivery into dendritic cells (D C) enhances CTL stimulation above other IL-12 applications: evidence for IL-12 intracrine activity in D C. Oncoimmunology 1: 847-855
5) Zhu H, Cao M, Straub K D, Hermonat P L. (2013) Systemic delivery of thiol-specific antioxidant hPRDX6 gene by AAV2/8 inhibits atherogenesis in LDLR KO mice on HCD. In press Gen Syndrom Gene Ther
6) Buchlis, G, Podsakoff G M, Radu A, Hawk S M, Flake A W, Mingozzi F, High K A. Factor IX expression in skeletal muscle of a severe hemophilia B patient 10 years after AAV-mediated gene transfer. Blood 119:3038-41, 2012.
7) Tratschin J D, Miller I L, Carter B J. Genetic analysis of adeno-associated virus: properties of deletion mutants constructed in vitro and evidence for an adeno-associated virus replication function. J Virol 51: 611-619, 1984
8) Sonntag F, Schmidt K, Kleinschmidt J A. A viral assembly factor promotes AAV2 capsid formation in the nucleolus. Proc Natl Acad Sci USA 107: 10220-10225, 2010.
9) Hermonat, P. L., Santin, A. D., DeRijcke, M., De Greves, J., Bishop, B., Han, L., Kokorina, N. (1999) Chromosomal latency and expression at map unit 96 of a wild-type plus adeno-associated virus vector(AAV)/Neo vector and identification of p81, a new AAV transcriptional promoter. J. Human Virology 2:359-368.
10) Srivastava A, Lusby E W, Berns K L Nucleotide sequence and organization of the adeno-associated virus 2 genome. J Virol 45: 555-564, 1983.
11) Meyers 2 Meyers, C., Mane, M., Kokorina, N., Alam, S., and Hermonat, P. L. (2000) Ubiquitous adeno-associated virus type 2 replicates in a model of normal skin. Virology 272:338-346
12) Meyers, C., Alam, S., Mane, M., and Hermonat, P. L. (2001) Altered biology of adeno-associated virus type 2 and human papillomavirus during dual infection of natural host tissue. Virology 287:30-39.
13) You, H., Liu, Y., Prasad, C. P., Agrawal, N., Zhang, D., Bandyopadhyay, S., Liu, H., Kay, H. H., Hermonat, P. L. (2006) Multiple human papillomavirus genes affect the adeno-associated virus life cycle. Virology 344(2):532-40.
14) Kang B Y, You H, Bandyopadhyay 5, Agrawal N, Melchert R B, Basnakian A G, Liu Y, Hermonat P L. (2009) Cervical cancer isolate PT3, super-permissive for adeno-associated virus replication, over-expresses DNA polymerase delta, PCNA, RFC and RPA. In press BMC Microbiology. 9:79.
15) Buller R M, Rose J A. Characterization of adeno-associated virus-induced polypeptides in K B cells. J Virol 25: 331-338, 1978.
16) Blacklow, N. R., M. D. Hoggan, and W. P. Rowe. Serologic evidence for human infection with adenovirus-associated viruses. J. Natl. Cancer Inst. 1968; 40:319-327.
17) Blacklow, N. R., M. D. Hoggan, M. S. Sereno, C. D. Brandt, H. W. Kim, R. H. Parrott, and R. M. Chanock. A seroepidemiologic study of adenovirus-associated virus infection in infants and children. Am. J. Epidemiol. 1971; 94:359-366.
18) Chirmule, N., K. Propert, S. Magosin, Y. Qian, R. Qian, and J. Wilson. 1999. Immune responses to adenovirus and adeno-associated virus in humans. Gene Ther. 6:1574-1583.
19) Shen X, Mizuguchi G, Hamiche A, Wu C. A chromatin remodeling complex involved in transcription and DNA processing. Nature 2000; 406: 541-544.
20) Wang M, Gu C, Qi T, Tang W, Wang L, Wang S, Zeng X. BAF53 interacts with p53 and functions in p53-mediated p21-gene transcription. J Biochem. 2007; 142: 613-620.
21) Lee K, Shim J H, Kang M J, Kim J H, Ahn J S, Yoo S J, Kim Kwon Y, Kwon H. Association of BAF53 with mitotic chromosomes. Mo Cells 2007; 24:288-293.
22) Chambers A L, Downs J A. The RSC and INO80 chromatin-remodeling compleses in DNA double-strand break repair. Prog Mol Biol Transl Sci 2012; 110: 229-261
23) Falbo K B, Shen X. Function of the INO80 chromatin remodeling complex in DNA replication. Front Biosci. 2012; 17:970-975.
24) Ren T, Dong W, Takahashi Y, Xiang D, Yuan Y, Liu X, Loughran T P Jr, Sun S C, Wang H G, Cheng H. HTLV-2 Tax immortalizes human CD4+ memory T lymphocytes by oncogenic activation and dysregulation of autophagy. J Biol Chem 2012; 287:34683-34693.
25) Ye J X, Xie L, Green P L. Tax and overlapping Rex sequences do not confer distinct transformation tropisms of human T-cell leukemia virus types 1 and 2. J Virol. 2003; 77: 7728-7735.
26) Ross T M, Narayan M, Fang Z Y, Minella A C, Green P L. Human T-cell leukemia type 2 tax mutants that selectively abrogate NFkappaB or CREB/ATF activation fail to transform primary T cells. J Virol 2000; 74:2655-2662.
27) Alunni-Fabbroni M, Littlewood T, Deleu L, Caldeira S, Giarre M, Orco M D, Tommasino M. Induction of S phase and apoptosis by human papillomavirus type 16 E7 protein are separable events in immortalized rodent fibroblasts. Oncogene 2000; 19: 2277-2285.
28) Hermonat, P L Inhibition of H-ras expression by the adeno-associated virus Rep78 transformation supressor gene product. Cancer Res. 1991; 51:3373-3377.
29) Hermonat, P. L. Adeno-associated virus inhibits human papillomavirus type 16: A viral interaction implicated in cervical cancer. Cancer Res 1994; 54:2278-2281.
30) Hermonat, P. L. Down-regulation of the human c-fos and c-myc proto-oncogene promoters by adeno-associated virus Rep78. Cancer Letters 1994, 81:129-136.
31) Hermonat P L, Plott R T, Santin A D, Parham G P, Flick J T. The adeno-associated virus Rep78 gene inhibits oncogenic transformation of primary keratinocytes by a human papillomavirus type 16-ras chimeric. Gynecologic Oncology 1997; 66:487-494.
32) Jiang H., Pierce G F, Ozelo M C, de Paula E V, Vargas J A, et al. (2006) Evidence of multiyear factor IX expression by AAV-mediated gene transfer to skeletal muscle in an individual with severe hemophilia B. Molec Ther 24: 452-455.
33) Sonntag F, Schmidt K, Kleinschmidt J A. A viral assembly factor promotes AAV2 capsid formation in the nucleolus. Proc Natl Acad Sci USA. 2010 Jun. 1; 107(22):10220-5.
34) You, H., Liu, Y., Carey, M. J., Lowery, C. L., and Hermonat, P. L. (2002) Defective 3A trophoblast-endometrial cell adhesion, and altered 3A growth and survival by human papillomavirus type 16 oncogenes. Molecular Cancer Research 1:25-31.
35) Chadeuf G, Ciron C, Moullier P, Salvetti. Evidence for encapsidation of prokaryotic sequences during recombinant adeno-associated virus production and their in vivo persistence after vector delivery. Mol Ther 2005; 12: 744-753.
36) Nony P, Chadeuf G, Tessier J, Moullier P, Salvetti A. Evidence for packaging rep-cap sequences into adeno-associated virus (AAV) type 2 capsids in the absence of inverted terminal repeats: a model for generation of rep-positive AAV particles. J Virol. 2003; 77: 778-781.
37) Gao G P, Wang Q, Wang L, et al. Inadvertent gene transfer of co-packaged rep and cap sequences during the production of AAV vector and its potential impact on vector performance. Mol Ther 2008; S105-S106.
38) Allay J A, Sleep S, Long S et al. Good manufacturing practice production of self-complementary serotype 8 adeno-associated viral vector for a hemophilia B clinical trial. Hum Gene Ther 2011; 22: 595-604.
39) Kim C M, Koike K, Saito 1. et al. HBx gene of hepatitis B virus induces liver cancer in transgenic mice. Nature. 1991; 351 1317-320
40) Bouchard M J, Schneider R J. The enigmatic X gene of hepatitis B virus. J Virol. 2004; 78:12725-12734
41) Sir D, Tian Y, Chen W L. et al. The early autophagic pathway is activated by hepatitis B virus and required for viral DNA replication. Proc Natl Acad Sci USA. 2010; 107:4383-4388
42) Chang L S, Pater M M, Hutchinson N1, di Mayorca G. Transformation by purified early genes of simian virus 40. Virology. 1984 March; 133(2):341-53.
43) zur Hausen H. Novel human polyomaviruses—re-emergence of a well known virus family as possible human carcinogens. Int J Cancer. 2008 Jul. 15; 123(2):247-50.
44) zur Hausen H. Papillomaviruses and cancer: from basic studies to clinical application. Nat Rev Cancer. 2002; 2:342-350
45) Narisawa-Saito M, Kiyono T. Basic mechanisms of high-risk human papillomavirus-induced carcinogenesis: roles of E6 and E7 proteins. Cancer Sci. 2007; 98:1505-1511
46) Suprynowicz F A, Disbrow G L, Krawczyk E, Simic V, Lantzky K, Schlegel R. HPV-16 E5 oncoprotein upregulates lipid raft components cavelin-1 and ganglioside GM1 at the plasma membrane of cervical cells. Oncogene. 2008; 27: 1071-1081.
47) Nevels M, Dobner T. Determination of the transforming activities of adenovirus oncogenes. Methods Mol Med. 2007; 131: 187-195.
48) Sarnow, P., Ho, Y. S., Williams, J. & Levine, A. J. (1982a). Adenovirus E1B-58 kD tumor antigen and SV40 large tumor antigen are physically associated with the same 54 kD cellular protein in transformed cells. Cell 28, 387-394.-A
49) Nleisch U, Fognani C, Babiss L E. Adenovirus E1A-p105(Rb) protein interactions play a direct role in the initiation but not the maintenance of the rodent cell transformed phenotype. Oncogene 1991 6:1031-1036.
50) Moore M, Horikoshi N, Shenk T. Oncogenic potential of adenovirus E4orf6 protein. PNAS USA 93:11295-11301.
51) Park J, Wood M A, Cole M D. BAF53 forms distinct nuclear complexes and functions as a critical c-Myc-interacting nuclear cofactor for oncogenic transformation. Mol Cell Biol. 2002 March; 22(5):1307-16.
52) Baidoobonso S M, Guidi B W, Myers L C. Med19(Rox3) regulates intermodule interactions in the Saccharomyces cerevisae mediator complex. J Biol Chem 2007; 282: 5551-5559.
53) Liu Y, Tao X, Fan L, Gu C, Feng Y. Knockdown of mediator 19 mediates H1299 lung adenocarcinoma cell clone conformation, growth, and metastasis. Asian Pac J Cancer Prev 2012; 13:3695-3700.
54) Werness B A, Levine A J, Howley P M. Association of human papillomavirus types 16 and 18 E6 proteins with p53. Science. 1990 Apr. 6; 248(4951):76-9.
55) Hermonat, P. L. (1989) The adeno-associated virus Rep78 gene inhibits cellular transformation induced by bovine papillomavirus. Virology 172:253-261.
56) Hermonat, P. L. (1994) Adeno-associated virus inhibits human papillomavirus type 16: A viral interaction implicated in cervical cancer. Cancer Research 54:2278-2281.
57) Labow, M. A., Hermonat, P. L., and Berns, K. l. (1986) Positive and negative autoregulation of the adeno-associated virus type 2 genome. J. Virology 60(1):251-258.
58) You C X, Liu Y, Shi M, Cao M, Luo R-C, Hermonat P L. (2010) Comparison of AAV/IL-7 autocrine (T cell) versus paracrine (DC) gene delivery for enhancing CTL stimulation and function. Cancer Imm & Immunotherapy. 59: 779-787.
59) Liu, Y., Li, D., Chen; J. Xie, J., Bandyopadhyay, S., Zhang, D., Nemarkommula, A. R., Liu, H., Mehta, J. L., Hermonat, P. L. (2006) Inhibition of atherogenesis in LDLR knockout mice by systemic delivery of adeno-associated virus type 2-hlL-10. Atherosclerosis 188: 19-27.
60) You, H., Liu, Y., Carey, M. J., Lowery, C. L., and Hermonat, P. L. (2002) Defective 3A trophoblast-endometrial cell adhesion, and altered 3A growth and survival by human papillomavirus type 16 oncogenes. Molecular Cancer Research 1:25-31.
61) Cao M, You H, Hermonat P L The X gene of adeno-associated virus 2 (AAV2) is involved in viral DNA replication. In press PLoS ONE 62) Gao G, Vandenberghe L H, Wilson J M. New recombinant serotypes of AAV vectors. Curr Gene Ther. 2005 June; 5(3):285-97
63) Kleinschmidt Sonntag F, Schmidt K, Kleinschmidt J A. (2010) A viral assembly factor promotes AAV2 capsid formation in the nucleolus. Proc Natl Acad Sci USA 107: 10220-10225.
64) Smith R H, Kotin R M. An adeno-associated virus (AAV) initiator protein, Rep78, catalyzes the cleavage and ligation of single-stranded AAV on DNA. J Virol.
2000 April; 74(7):3122-9.

All cited patents, patent documents, and other references are incorporated by reference.

What is claimed is:

1. A therapeutic composition comprising:
a plurality of recombinant adeno-associated virus (AAV) virus particles comprising native AAV DNA and recombinant therapeutic DNA,
wherein none of the AAV virus particles has an active AAV X gene.

2. An engineered eukaryotic host cell comprising:
a chromosomally integrated X expression cassette comprising an AAV X gene under expression control of a promoter effective to express the X gene in the host cell;
wherein the host cell is in vitro.

3. The engineered eukaryotic host cell of claim 2 wherein the host cell is a HEK293 derivative.

4. The engineered eukaryotic host cell of claim 2 wherein the promoter for the chromosomally integrated AAV X gene is not a native AAV X gene promoter.

5. The engineered eukaryotic host cell of claim 2 wherein the promoter is cytomegalovirus (CMV) immediate early promoter (CMV promoter).

6. The engineered eukaryotic host cell of claim 4 wherein the promoter effective to express the X gene in the cell gives higher expression in the host cell than the native X gene promoter.

7. An expression system for producing recombinant AAV virus particles, the expression system comprising:
a eukaryotic host cell comprising a chromosomally integrated AAV X expression cassette comprising an AAV X gene under expression control of a promoter effective to express the X gene in the host cell;
one or more AAV helper expression cassettes collectively encoding and expressing AAV rep and cap proteins and other AAV helper proteins;
an insert replication cassette encoding an insert nucleic acid flanked by inverted terminal repeats for packaging into recombinant AAV virus particles;
wherein none of the AAV helper or insert expression or replication cassettes comprises an active AAV X gene.

8. The expression system of claim 7 wherein the chromosomally integrated X expression cassette comprises a promoter that controls X expression and is not the native X promoter and is a stronger promoter in the host cell than the native X promoter.

9. The expression system of claim 7 wherein the chromosomally integrated X expression cassette is not a part of a full active chromosomally integrated cap gene.

10. The expression system of claim 7 wherein the one or more other AAV helper proteins comprises lip.

11. The expression system of claim 7 wherein the one or more other AAV helper proteins comprises lip and cap.

12. The expression system of claim 7 wherein the one or more other AAV helper proteins comprises only native host cell proteins.

13. A method of producing recombinant AAV virus particles comprising:
expressing AAV X gene from a chromosomally integrated X gene in a eukaryotic host cell;
expressing AAV rep and cap genes in the host cell;
expressing AAV helper genes other than X, rep and cap, in the host cell;
replicating a recombinant construct comprising a recombinant gene of interest flanked by AAV inverted terminal repeats in the host cell; and
packaging the replicated recombinant construct into recombinant AAV virus particles.

14. The method of claim 13 further comprising purifying the recombinant AAV virus particles.

15. The method of claim 13 wherein the chromosomally integrated X gene is expressed from a promoter that is not a native AAV X gene promoter.

16. The method of claim 13 wherein the chromosomally integrated X gene is not a part of a full active chromosomally integrated cap gene.

17. The method of claim 13 wherein the other helper genes comprise lip and cap.

18. A method of producing recombinant AAV virus particles comprising:
expressing AAV X gene in a eukaryotic host cell from a promoter that is not a native AAV X gene promoter and is more active in the host cell than the native AAV X gene promoter;
expressing AAV rep and cap genes in the host cell;
expressing AAV helper genes other than X, rep, and cap in the host cell;
replicating a recombinant construct comprising a recombinant gene of interest flanked by AAV inverted terminal repeats in the host cell; and
packaging the replicated recombinant construct into recombinant AAV virus particles.

19. An isolated plasmid comprising AAV cap gene, wherein the plasmid does not comprise an active AAV X gene.

20. A eukaryotic host cell comprising:
an expression cassette comprising AAV gene X under the control of a promoter, wherein the promoter is not a native AAV promoter;
wherein the eukaryotic host cell is ex vivo.

21. The eukaryotic host cell in vitro of claim 16 wherein the promoter is more active in the host cell than the native AAV X gene promoter.

22. The eukaryotic host cell in vitro of claim 16 wherein the promoter is CMV promoter.

23. An expression system for producing recombinant AAV virus particles, the expression system comprising:
one or more AAV helper expression cassettes collectively encoding and expressing AAV rep and cap proteins and other AAV helper proteins; and
an insert replication cassette encoding an insert nucleic acid flanked by inverted terminal repeats for replication and packaging into recombinant AAV virus particles;
wherein none of the AAV helper or insert expression or replication cassettes comprises an active AAV X gene.

24. The expression system of claim 19 further comprising a eukaryotic host cell, wherein the eukaryotic host cell comprises the one or more AAV helper expression cassettes and the insert replication cassette.

25. The expression system of claim 20 wherein the eukaryotic host cell does not comprise an active AAV X gene.

26. A method of producing recombinant AAV virus particles comprising:
expressing AAV rep and cap genes in a host cell;
expressing AAV helper genes other than X, rep, and cap in the host cell;
replicating a recombinant construct comprising a recombinant gene of interest flanked by AAV inverted terminal repeats in the host cell; and
packaging the recombinant construct into recombinant AAV virus particles;
wherein the host cell does not comprise an active AAV X gene and the method therefore does not comprise expressing an active AAV X gene in the host cell.

27. The method of claim 26 wherein the host cell is in vitro.

28. The method of claim 27 wherein the host cell is a HEK293 derivative.
